SEQUENCE LISTING (52 sequences)

SEQ ID NO: 1 - DNA, 19 bp, Artificial Sequence
  Synthetic primer.
tcgttcatctgccgcatga

SEQ ID NO: 2 - DNA, 21 bp, Artificial sequence
  Synthetic primer.
ttcacagagccaagcccattc

SEQ ID NO: 3 - DNA, 20 bp, Artificial sequence
  Synthetic primer.
aaccttcccgcagctaacag

SEQ ID NO: 4 - DNA, 20 bp, Artificial sequence
  Synthetic primer.
agtcctctttgggccacctt

SEQ ID NO: 5 - DNA, 21 bp, Artificial sequence
  Synthetic primer.
ggcgttgttgattggactagg

SEQ ID NO: 6 - DNA, 21 bp, Artificial sequence
  Synthetic primer.
gaatggagtctccaacaccca

SEQ ID NO: 7 - DNA, 21 bp, Artificial sequence
  Synthetic primer.
aggaggacagatcccaatgga

SEQ ID NO: 8 - DNA, 21 bp, Artificial sequence
  Synthetic primer.
gcaaccttctggatgccttct

SEQ ID NO: 9 - DNA, 21 bp, Artificial sequence
  Synthetic primer.
agctgatgtgttcccaaggct

SEQ ID NO: 10 - DNA, 20 bp, Artificial sequence
  Synthetic primer.
cataatggctgcatcccgtt

SEQ ID NO: 11 - DNA, 21 bp, Artificial sequence
  Synthetic primer.
ggtggagagcaccaagacaga

SEQ ID NO: 12 - DNA, 19 bp, Artificial sequence
  Synthetic primer.
gccggagtcgacaatgatg

SEQ ID NO: 13 - DNA, 22 bp, Artificial sequence
  Synthetic primer.
ggaactgaagcctcaaccaatc

SEQ ID NO: 14 - DNA, 21 bp, Artificial sequence
  Synthetic primer.
ctccggctccagtacttctca

SEQ ID NO: 15 - DNA, 21 bp, Artificial sequence
  Synthetic primer.
acgcagatgttcgagtggaaa

SEQ ID NO: 16 - DNA, 19 bp, Artificial sequence
  Synthetic primer.
cgcccatgtcaagtgcatt

SEQ ID NO: 17 - DNA, 24 bp, Artificial sequence
  Synthetic primer.
ccagattggtggaggttactgagt

SEQ ID NO: 18 - DNA, 24 bp, Artificial sequence
  Synthetic primer.
gcgagagtcttcttggagcagtag

SEQ ID NO: 19 - DNA, 21 bp, Artificial sequence
  Synthetic primer.
agaacgcggatatgtttgctg

SEQ ID NO: 20 - DNA, 21 bp, Artificial sequence
  Synthetic primer.
atctaagccgctgcacacact

SEQ ID NO: 21 - DNA, 20 bp, Artificial sequence
  Synthetic primer.
ccgcccctacagtcagaaag

SEQ ID NO: 22 - DNA, 20 bp, Artificial sequence
  Synthetic primer.
gccccacgtgcttaaatcct

SEQ ID NO: 23 - DNA, 23 bp, Artificial sequence
  Synthetic primer.
ccctggactccaataacaacaca

SEQ ID NO: 24 - DNA, 22 bp, Artificial sequence
  Synthetic primer.
gccattggagctgtcactgtag

SEQ ID NO: 25 - DNA, 19 bp, Artificial sequence
  Synthetic primer.
ggaacggaccgtcaccttt

SEQ ID NO: 26 - DNA, 19 bp, Artificial sequence
  Synthetic primer.
tcccctgctcccattgagt

SEQ ID NO: 27 - DNA, 23 bp, Artificial sequence
  Synthetic primer.
agaaccacgataacccatgaaag

SEQ ID NO: 28 - DNA, 23 bp, Artificial sequence
  Synthetic primer.
gacttcagcctctcatccatcaa

SEQ ID NO: 29 - DNA, 22 bp, Artificial sequence
  Synthetic primer.
aggcatctgaattcccttctga

SEQ ID NO: 30 - DNA, 24 bp, Artificial sequence
  Synthetic primer.
agtccccaaatgccatttatttag

SEQ ID NO: 31 - DNA, 21 bp, Artificial sequence
  Synthetic primer.
gagacgtgaccggattaacga

SEQ ID NO: 32 - DNA, 21 bp, Artificial sequence
  Synthetic primer.
ccagaaccactgctttttcca

SEQ ID NO: 33 - DNA, 20 bp, Artificial sequence
  Synthetic primer.
ggagatggcacaggaggaaa

SEQ ID NO: 34 - DNA, 26 bp, Artificial sequence
  Synthetic primer.
cgtagtgcttcagtttgaagttctca

SEQ ID NO: 35 - DNA, 19 bp, Artificial sequence
  Synthetic primer.
gtttgccaaacacatcccg

SEQ ID NO: 36 - DNA, 19 bp, Artificial sequence
  Synthetic primer.
aagcaaagcgcaccatcag

SEQ ID NO: 37 - DNA, 21 bp, Artificial sequence
  Synthetic primer.
aacaagcaaatcgagtgcacc

SEQ ID NO: 38 - DNA, 21 bp, Artificial sequence
  Synthetic primer.
tccatagtggaatcctgacgc

SEQ ID NO: 39 - DNA, 21 bp, Artificial sequence
  Synthetic primer.
gtaccaacatgcaacgcaatg

SEQ ID NO: 40 - DNA, 21 bp, Artificial sequence
  Synthetic primer.
tgtgtatggattggtggcacc

SEQ ID NO: 41 - DNA, 21 bp, Artificial sequence
  Synthetic primer.
acccttcctcaacaccaacca

SEQ ID NO: 42 - DNA, 19 bp, Artificial sequence
  Synthetic primer.
atgcgtgtccgttgttcca

SEQ ID NO: 43 - DNA, 21 bp, Artificial sequence
  Synthetic primer.
atctagccaggcatgcagttg

SEQ ID NO: 44 - DNA, 20 bp, Artificial sequence
  Synthetic primer.
gctccaatctgcatcaagca

SEQ ID NO: 45 - DNA, 21 bp, Artificial sequence
  Synthetic primer.
ctggataagcacttggaacgg

SEQ ID NO: 46 - DNA, 21 bp, Artificial sequence
  Synthetic primer.
agacaaccaaagcgcaggtag

SEQ ID NO: 47 - DNA, 21 bp, Artificial sequence
  Synthetic primer.
tggaggcattagatggcttca

SEQ ID NO: 48 - DNA, 19 bp, Artificial sequence
  Synthetic primer.
atccgacggtaaatgccca

SEQ ID NO: 49 - DNA, 21 bp, Artificial sequence
  Synthetic primer.
attccgcctaaccccgtatgt

SEQ ID NO: 50 - DNA, 21 bp, Artificial sequence
  Synthetic primer.
gccgcgtagtgaaaatcctct

SEQ ID NO: 51 - DNA, 21 bp, Artificial sequence
  Synthetic primer.
agatgtcctggcgtcttctca

SEQ ID NO: 52 - DNA, 21 bp, Artificial sequence
  Synthetic primer.
tcataccgtgcagctctttgg

(19) United States
(12) Patent Application Publication
(10) Pub. No.: US 2009/0202659 A1
(43) Pub. Date: Aug. 13, 2009

(54) Modulation of Peripheral Clocks in Adipose Tissue

(76) Inventor: Jeffrey M. Gimble, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/916,848

(22) PCT Filed: Jun. 9, 2006
(86) PCT No.: PCT/US2006/022454

§ 371 Date: Oct. 10, 2008

Related U.S. Application Data
(60) Provisional application No. 60/689,315, filed on Jun. 10, 2005.

(51) Int. Cl. A61K 33/14; A61K 38/28; A61K 31/135; A61K 31/19; A61K 31/4164; A61K 38/10; A61K 38/16; A61K 38/08; A61K 31/60; C12Q 1/68; A61P 3/00

(52) U.S. Cl. 424/663; 514/3; 514/653; 514/557; 514/400; 514/14; 514/12; 514/16; 514/165; 435/6

(57) ABSTRACT

Genes encoding the transcription factors controlling the core circadian oscillator (BMAL, Clock, NPAS, Per) and their regulatory targets (Rev-erbα, Rev-erb) have been found in adipose tissue. The circadian pattern of these genes was entrained using restricted feeding. The circadian gene expression profiles were examined in mice and in undifferentiated and adipocyte-differentiated human adipose stem cells following exposure to nuclear hormone receptor ligands (dexamethasone or thiazolidinedione) or 30% fetal bovine serum. All three agents induced the initiation of a cyclic expression profile in representative circadian genes in the human adipose stem cells. The circadian genes studied displayed an oscillatory expression profile, characterized by both a zenith and nadir within a 24-28 hr phase. The circadian gene pattern has been lengthened with use of an inhibitor of glycogen synthase kinase 3 beta. Modulation of the circadian pattern to lengthen or shorten can be used to affect weight gain or loss, respectively.

The benefit of the filing date of provisional U.S. application Ser. No. 60/689,315, filed 10 Jun. 2005, is claimed under 35 U.S.C. § 119(e).

### TECHNICAL FIELD

This invention pertains to methods to entrain the peripheral clock in adipose tissue to treat diseases associated with weight gain or loss, for example, obesity, diabetes, immune dysfunctional diseases, cachexia related to cancer and AIDS, and metabolic disorders such as anorexia nervosa, bipolar disorders, and Prater-Willi Syndrome.

### BACKGROUND ART

Obesity is a condition of epidemic proportions in the United States, where over 50% of adults exceed the recommended body mass index (BMI) based on their height and weight. Associated with this rise in obesity is an increased incidence of diabetes mellitus in both the pediatric and adult populations. In a similar manner, large numbers of patients present annually with eating and sleep related disorders, such as nocturnal binge eating, anorexia nervosa, and bipolar disorders. Also, patients with cancer or AIDS frequently present with severe weight loss, muscle wasting, and loss of adipose tissue stores. In all of these disorders, dysregulated metabolic function plays a role.

Energy metabolism and adipose tissue function display distinct features linked to the diurnal light/dark cycle and reflect a circadian rhythm. Examples include the core body temperature, which varies during the day in a rhythmic manner and the secretion of proteins by adipose tissue and other organs, including but not limited to, leptin, adiponectin, PAI-1, angiotensinogen, and lipoprotein lipase, which have important roles in regulating cardiovascular function and cardiovascular disease risk factors. In addition, transcription factors controlling key adipocyte functions, including but not limited to, SREBP/ADD1 and PPARα, display a circadian pattern of expression in liver, heart, and other tissues. It is well established that the brain's central clock, located with the suprachiasmatic nucleus (SCN), plays a major role in coordinating these events; however, there is growing evidence that peripheral clocks, located within distinct tissues, also operate.

Circadian rhythms in gene expression synchronize biochemical processes with the external environment, allowing the organism to function effectively in response to constantly changing physiological challenges (R. Allada et al., 2001; U.S. Patent Application Publication No. 2002/0151590). Genes belonging to the basic helix-loop-helix/Per-Arnt-Simpleminded (bHLH-PAS) domain family, encoded by Clock (or its paralog Npas2), Bmal1, Period (Per), and Cryptochrome (Cry) genes, play a central role in this process (R. Allada et al., 2001). Heterodimers of CLOCK and BMAL1 drive the transcription of Per and Cry (N. Gekakis et al, 1998; T. K. Darlington et al., 1998; U.S. Patent Application Publication No. 2003/0059848). After translation in the cytoplasm, PER and CRY proteins heterodimerize, translocate to the nucleus, and regulate the activity of CLOCK:BMAL1, completing a transcriptional/translational feedback loop (J. A. Ripperger et al., 2000; and E. A. Griffin, Jr., et al, 1999). As a consequence, expression levels for these two sets of genes display anti-phasic oscillatory profiles with respect to one another. In addition, CLOCK:BMAL1 heterodimers regulate the transcription of circadian effector genes, including those encoding the transcription factors DBP (albumin D binding protein) and REV-ERBα, implicated in multiple physiologic functions (J. A. Ripperger et al., 2000; and A. Balsalobre et al., 1998).

Recent work using the nzPer2 promoter:Luciferase reporter in mice has demonstrated a persistent oscillatory Luciferase profile for >20 days ex vivo, not just in the core circadian oscillator in the suprachiasmatic nucleus (SCN), but also in liver and muscle explants (S. H. Yoo et al., 2004). These peripheral oscillators continue to operate even in animals where the SCN has been surgically ablated, demonstrating that independent circadian oscillators operate within peripheral tissues. Several in vitro studies of fibroblast cell lines further support these findings. Circadian gene expression of Clock, Per, Dbp, and Bmal1 in these cells can be induced with exposure to dexamethasone, high serum concentrations, or glucose (E. Nagoshi et al., 2004). In addition, a method has been proposed to alter a patient's circadian rhythm by treatment with corticotrophin releasing factor antagonists (U.S. Pat. No. 6,432,989). Also, recent studies have extended these analyses to human dermal fibroblasts. When transduced with a Bmal1 promoter/luciferase reporter construct and induced with dexamethasone, the luciferase activity in the human fibroblast cells showed circadian rhythms of 24.5 hr (S. A. Brown et al., 2005). While the circadian cycle for most donors clustered between 24-25 hr, the range extended from 22.75 to 26.25 hr among 19 donors.

In a number of human disease states, changes in circadian rhythms are associated with altered adipose tissue function. For example, when bipolar patients receive pharmacotherapy, such as lithium chloride, they gain weight rapidly and become obese (A. Fagiolini et al., 2002, 2003). The clinical hallmarks of bipolar disorder include abnormal sleep patterns and disordered circadian function. Recent findings have linked lithium chloride's inhibitory effects on glycogen synthase kinase 3 to the regulation of Rev-erbα (L. Yin et al., 2006). Likewise, the serum levels of many adipokines, such as TNFα, IL-6, adiponectin, leptin, and PAI-1, exhibit strong circadian patterns (A. Gavrila et al., 2003a, 2003b; S. E. la Fleur et al., 2001; J. G. van der Bom, et al., 2003; A. N. Vgontzas et al., 1999; A. N. Vgontzas et al., 2002; and A. N. Vgontzas et al., 2004a, 2004b). Epidemiological studies have correlated early morning peaks in the circulating levels of PAI-1 and the incidence of myocardial infarction, sudden death, and heart failure in the general patient population (A. J. Stunkard et al., 2004; G. S. Birketvedt et al., 1999). Consistent with this is the observation that the PAI-1 promoter contains DNA response elements recognized by the CLOCK:BMAL1 dimers (J. G. van der Bom et al., 2003). Interestingly, patients diagnosed with obesity and type 2 diabetes fail to display circadian variability in the incidence of myocardial infarction (J. S. Rana et al., 2003). These same patients display chronically elevated levels of PAI-1, resulting in a dampening of the circadian variation in its expression (J. G. van der Bom et al., 2003). Similarly, epidemiological studies show an increased incidence of metabolic syndrome among night-shift workers, whose activity period is reversed relative to the light:dark period (U. Holmback et al., 2003).

The symptoms of arthritic diseases have been shown to display a circadian pattern (N. G. Arvidson et al., 1997; M. Cutolo et al., 2003). The timing of treatment with glucocorticoids was shown to have a significant effect on the severity of morning stiffness in patients with rheumatoid arthritis (N. G. Arvidson et al., 1997). Also, timing of administering agents to alter the prolactin rhythm in humans has been proposed as a treatment of rheumatoid arthritis (U.S. Pat. No. 5,905,083).

The suprachiasmatic nucleus and other sites within the central nervous system play a major role in controlling circadian rhythms, acting both directly through sympathetic nervous innervation of target organs and, indirectly, through the release of glucocorticoids and other circulating systemic factors (P. L. Lowrey et al., 2004). Nevertheless, recent evidence suggests that peripheral tissues may possess some degree of circadian autonomy (S. H. Yoo et al., 2004; L. D. Wilsbacher et al., 2002). Isolated rat cardiomyocytes exhibited an intrinsic circadian apparatus activated following an exposure to serum shock or norepinephrine, but not glucose (D. J. Durgan et al., 2005). Microarray and qRT-PCR analyses have shown that the liver, heart, and other tissues express genes encoding the circadian transcriptional apparatus (S. Panda et al., 2002b; K. F. Storch et al., 2002; H. R. Ueda et al., 2002; M. E. Young, 2006; M. E. Young et al., 2002). The relative levels of these genes fluctuated in a diurnal manner and up to 10% of the transcriptome clustered with them in a temporally parallel expression profile (S. Panda et al., 2002b; K. F. Storch et al., 2002; H. R. Ueda et al., 2002). Moreover, liver, heart, and muscle isolated from mice transgenic for a Per2 promoter/luciferase reporter construct displayed independent oscillatory expression of the reporter gene, maintaining a circadian rhythm in the absence of SCN input for up to 20 days (S. H. Yoo et al., 2004).

There is a growing body of literature implicating the circadian transcriptional regulatory apparatus in regulating adipose metabolism. In vitro transfection studies have determined that the PAS domain family members, BMAL1 and EPAS2, are adiopogenic transcriptional regulators. (S. Shimba et al., 2005; S. Shimba et al., 2004) Likewise, Rev-erba is both a transcriptional target as well as an adipogenic regulator. Recent studies indicate that glycogen synthase kinase 31-mediated mechanisms control Rev-erbα (L. Yin et al., 2006). In vivo studies indicate that the loss of BMAL1 and CLOCK function is associated with a loss of the diurnal rhythmicity of circulating glucose and triglyceride levels in murine models (R. D. Rudic et al., 2004). Moreover, mice with mutations in the core circadian regulator Clock lose their diurnal feeding behavior as they become hyperphagic, obese, and subject to the morbidities associated with the metabolic syndrome (F. W. Turek et al., 2005).

A number of systemic features reflect the diurnal cycle and circadian rhythm of the organism (Czeisler et al 1999). Some of the best characterized in man are body temperature, melatonin levels, and glucocorticoid levels which demonstrate a distinct zenith (peak) and nadir during a 24 hour period that is well conserved among individuals. There are additional proteins that have been shown to display an expression profile consistent with a diurnal variation. Some of these are summarized in the following table:

| | Reference |
|---|---|
| A. Adipose Related Genes - | |
| Secreted | |
| Leptin | A. Elimam et al., 1998; |
| | A. Kalsbeek et al., 2001 |
| Lipoprotein Lipase | A. Benavides et al., 1998 |
| Angiontensinogen | C. S. Narayanan et al., 1998; |
| | Y. Naito et al., 2002, 2003; |
| Adiponectin | A. Gavrila et al., 2003a, 2003b |
| PAI-1 | K. Maemura et al., 2000; |
| (Plasminogen activator inhibitor I) | J. A. Shoenhard et al., 2003; |
| | Y. Naito et al., 2003; |
| | T. Mohri et al., 2003 |
| Interleukin 6 | A. N. Vgontzas et al., 1999, 2002 |

B. Adipose Related Genes -
Transcription Factors

| | |
|---|---|
| Peroxisome proliferator | T. Lemberger et al., 1996 |
| activated receptor α (PPARα) | |
| Sterol response element | D. D. Patel et al., 2001 |
| binding protein (SREBP) | |

The relationship of the central circadian clock to these peripheral systems remains to be fully defined. There is evidence that the peripheral tissues may operate to some in balance with the central clock and may work to balance and/or offset it. This may account for the discomfort frequently associated with long distant travel (jet-lag), independent of sleep deprivation. The peripheral clocks act in concert with the central clock to reset the body's circadian rhythm.

While circadian dysfunction and disease pathogenesis are clearly linked, the existence of a peripheral clock and the role of circadian genes in adipose tissue physiology had not been noted until this current invention. However, in a paper published after the Jun. 10, 2005 priority date, but before the filing of this application, the gene expression of certain clock genes were reported to show a circadian pattern in mouse periogonadal adipose tissue (H. Ando et al., 2005).

### DISCLOSURE OF INVENTION

I have discovered that the genes encoding the transcription factors controlling the core circadian oscillator (BMAL, Clock, NPAS, Per) and their regulatory targets (Rev-erba, Rev-erb) are found in adipose tissue. The circadian pattern of these genes can be entrained using restricted feeding. The circadian gene expression profiles were examined in mice and in undifferentiated and adipocyte-differentiated human ASCs following exposure to nuclear hormone receptor ligands (dexamethasone or thiazolidinedione) or 30% fetal bovine serum. All three agents induced the initiation of a cyclic expression profile in representative circadian genes. The response to fetal bovine serum preceded that of the nuclear hormone receptor ligands by ˜4 hours. Likewise, the response of adipocyte-differentiated cells to the inductive agents was accelerated relative to their donor-matched undifferentiated controls. Overall, the circadian genes studied displayed an oscillatory expression profile, characterized by both a zenith and nadir within a 24-28 hr phase. Furthermore, individual donors displayed variation in the phase length of circadian gene expression. Thus, I have demonstrated that nuclear hormone receptor ligands influence the circadian transcriptional apparatus within the human adipose tissue, and that responses to these compounds can vary as a function of cell differentiation. The circadian pattern has also been lengthened with use of an inhibitor of glycogen synthase kinase 3 beta. Modulation of the circadian pattern by lengthening or shortening can be used to affect weight gain or loss, respectively.

### BRIEF DESCRIPTION OF DRAWINGS

FIG. 1 illustrates the circadian oscillator gene expression patterns for eight genes (Npas2, Bmal1, Clock, Per1, Per2, Per3, Cry1, and Cry2) as determined by quantitative RT-PCR analysis on RNA from four peripheral tissues (liver, brown adipose tissue (BAT), inguinal adipose tissue (iWAT), and epididymal adipose tissue (eWAT)) isolated from mice every 4 hr over a 48-hr time period. All values are reported as averages±S.D.

FIG. 2 illustrates the circadian oscillator gene expression patterns for seven genes (Rev-erbα, Rev-erbβ, Arnt, StraI3, Dbp, E4bp4, and Id2) as determined by quantitative RT-PCR analysis on RNA from four peripheral tissues (liver, brown adipose tissue (BAT), inguinal adipose tissue (iWAT), and epididymal adipose tissue (eWAT)) isolated from mice every 4 hr over a 48-hr time period. All values are reported as averages±S.D.

FIG. 3 illustrates the circadian oscillation of serum biomarkers (corticosterone, melatonin, and leptin) as measured from pooled mice serum every 4 hr over a 48-hr period. All values are reported as averages±S.D.

FIG. 4 illustrates the number of overlapping periodically expressed genes in liver, BAT, and iWAT as determined by microarray analysis, with periodicity detected by discrete Fourier transform.

FIG. 5 illustrates the circadian oscillator gene expression patterns for eight genes (Npas2, Bmal1, Clock, Per1, Per2, Per3, Cry1, and Cry2) as determined by quantitative RT-PCR analysis on RNA from four peripheral tissues (liver, brown adipose tissue (BAT), inguinal adipose tissue (iWAT), and epididymal adipose tissue (eWAT)) isolated from two groups of mice every 4 hr over a 24-hr time period. The control group (dashed line) had unrestricted feeding, and the restricted feeding group (solid line) was fed only at night. All values are reported as averages±S.D.

FIG. 6 illustrates the circadian oscillator gene expression patterns for five genes (Rev-erbα, Rev-erbβ, Dbp, E4bp4, and Id2) as determined by quantitative RT-PCR analysis on RNA from four peripheral tissues (liver, brown adipose tissue (BAT), inguinal adipose tissue (iWAT), and epididymal adipose tissue (eWAT)) isolated from two groups of mice every 4 hr over a 24-hr time period. The control group (dashed line) had unrestricted feeding, and the restricted feeding group (solid line) was fed only at night. All values are reported as averages±S.D.

FIG. 7 illustrates the effect of restricted feeding on the daily oscillation pattern of serum corticosterone (measured in serum collected every 4-h over a 24-hr time period), and on the daily food intake and body weight measured over 7 days. The control group (dashed line) had unrestricted feeding, and the restricted feeding group (solid line) was fed only at night. All values are reported as averages±S.D.

FIG. 8A illustrates the circadian oscillator gene expression patterns for eight genes (Npas2, Cry1, Cry2, Per1, Rev-erbα, Npas2, Per3, and Rev-erbβ) as determined by quantitative RT-PCR analysis on RNA from undifferentiated human adipose stem cells exposed to fresh serum medium alone. Cells were harvested every 4 hr over a 48-hr time period. Each graph represents an individual human donor.

FIG. 8B illustrates the circadian oscillator gene expression patterns for eight genes (Npas2, Cry1, Cry2, Per1, Rev-erbα, Npas2, Per3, and Rev-erbβ) as determined by quantitative RT-PCR analysis on RNA from undifferentiated human adipose stem cells exposed for 2 hr to dexamethasone (1 μM) at time), and then maintained in serum free medium alone. Cells were harvested every 4 h over a 48-hr time period. Each graph represents an individual human donor.

FIG. 9 illustrates the circadian oscillator gene expression patterns for four genes (Bmal1, Per3, Rev-erbα, and Rev-erbβ) as determined by quantitative RT-PCR analysis on RNA from undifferentiated human adipose stem cells and differentiated human adipose stem cells from three donors (each line is an individual donor), when the cells were exposed for 2 hr to medium supplemented with 30% bovine serum albumin, and then maintained in serum free medium alone for 48 hr. Cells were harvested every 4 h over a 48-hr time period. All values are reported as averages±S.D. [0025]

FIG. 10 illustrates the circadian oscillator gene expression patterns for four genes (Bmal1, Per3, Rev-erbα, and Rev-erbβ) as determined by quantitative RT-PCR analysis on RNA from undifferentiated human adipose stem cells and differentiated human adipose stem cells from three donors (each line is an individual donor), when the cells were exposed for 2 hr to medium supplemented with dexamethasone (1 μM), and then maintained in serum free medium alone for 48 hr. Cells were harvested every 4 h over a 48-hr time period. All values are reported as averages±S.D.

FIG. 11 illustrates the circadian oscillator gene expression patterns for four genes (Bmal1, Per3, Rev-erbα, and Rev-erbβ) as determined by quantitative RT-PCR analysis on RNA from undifferentiated human adipose stem cells and differentiated human adipose stem cells from three donors (each line is an individual donor), when the cells were exposed for 2 hr to medium supplemented with rosiglitazone (5 uM), and then maintained in serum free medium alone for 48 hr. Cells were harvested every 4 h over a 48-hr time period. All values are reported as averages±S.D.

FIG. 12 illustrates the circadian oscillator gene expression patterns for three genes (Bmal1, Per3, and Rev-erba) as determined by quantitative RT-PCR analysis on RNA from adipocyte-differentiated human adipose stem cells exposed for 2 hours to serum-free medium supplemented with 1 μM dexamethasone and then converted to either serum-free media (SF), or serum-free media with 30 μM SB415286 (SB415; an inhibitor of glycogen synthase kinase 3 beta (GSK3β) for the time indicated in the figure. Assays were performed in triplicate and values displayed are the mean±S.D.

The present invention provides methods for the use of circadian rhythm-related “clock” genes in adipose tissue as targets in the treatment of metabolic disorders. I have shown that the genes encoding the circadian transcriptional apparatus exhibit an oscillatory expression profile in murine brown adipose tissue and subcutaneous and visceral white adipose tissue. Environmental stimuli such as temporal restriction of food access were shown to phase-shift the expression of these genes by up to 8 hrs in murine tissues. In addition, at least 20-25% of the murine adipose tissue transcriptome displayed an oscillatory expression profile. I have also shown that isolated human adipose stem cells have the potential to serve as a surrogate in vitro model for analysis of circadian mechanisms in human adipose tissue. The temporal kinetics of circadian gene induction in human ACSs changed as a function of their differentiation status. Mature adipocytes differ from preadipocytes with respect to their response to nuclear hormone receptor ligands, such as corticosterone or exogenous medications, such as oral anti-diabetic agents. The time of day that thiazolidinediones are administered to diabetic patients may have a significant impact on their therapeutic effects. Knowing the circadian pattern of the peripheral clock in adipose tissue could help determine the most efficacious time to administer certain medications. In one aspect of the present invention, methods for the use of “clock” genes as targets in the treatment and prevention of obesity or other weight gain is explored. In another aspect of the invention, methods for the use of “clock” genes as targets or indicators on how to treat diabetes mellitus. In another aspect of the invention, methods for the use of “clock” genes as targets in the treatment of eating disorders, including but not limited to, anorexia nervosa and nocturnal binge eating. In yet another aspect of the invention, methods for the use of “clock” genes as targets in the treatment of nutritional deficiency states, including but not limited to, cancer cachexia and wasting syndromes in patients with Acquired Immune Deficiency Syndrome (AIDS). Other objects and features of the invention will be more fully apparent from the following disclosure and appended claims.

In one embodiment of the invention, “clock” genes are demonstrated to be expressed in adipose tissue depots in a circadian manner. In another embodiment of the invention, the adipose tissue peripheral “clock” genes are entrained by administration of exogenous agents, including but not limited to, a glycogen synthase kinase 3, inhibitor, glucocorticoids and thiazolidinediones. In another embodiment of the invention, the adipose tissue peripheral “clock” genes are entrained by alteration of feeding schedule. In yet another embodiment of the invention, obesity is prevented or ameliorated by manipulation of the adipose tissue peripheral “clock” genes by the use of exogenous agents, including but not limited to glucocorticoids, thiazolidinediones, thyroid hormone, and other nuclear hormone receptor ligands.

### DEFINITIONS

CIRCADIAN RHYTHM refers to the diurnal rhythm of events and biochemical phenomenon displayed by living organisms. These events are routinely coordinated by the light/dark cycle of the day and are centrally regulated in mammals through the suprachiasmatic nucleus.

PAS FAMILY refers to a family of proteins with homology to a domain found in the Periodic/ARNT (Aryl hydrocarbon nuclear transporter)/Sim proteins. The PAS domain is associated with the Clock genes involved in regulating circadian rhythms.

HLH FAMILY refers to the basic Helix Loop Helix family of proteins. The bHLH serves to promote heterodimerization between transcriptional regulatory proteins.

CLOCK refers to a PAS domain/bHLH protein that heterodimerizes with BMAL1 to form a transcription complex that positively regulates the circadian clock in the surprachiasmatic nucleus (SCN).

BMAL-1 refers to a PAS domain/bHLH protein that heterodimerizes with CLOCK to form a transcription complex that positively regulates the circadian clock in the SCN and dimerizes with NPAS2 and other proteins in other areas of the brain and peripheral tissues.

NPAS2 refers to a PAS domain/bHLH protein that heterodimerizes with BMAL1 to form a transcription complex that positively regulates the circadian clock in the brain and other tissues.

PER refers to the Periodic protein(s) which are regulated by the BMAL1/CLOCK complex. The PER protein acts with CRY to form a negative transcriptional regulatory complex that oscillates in expression with CLOCK and NPAS2. The PER proteins are expressed in both central and peripheral clock tissues.

CRY refers to the Cryptochrome protein(s) which are regulated by the BMAL1/CLOCK complex. The CRY protein acts with PER to form a negative transcriptional regulatory complex that oscillates in expression with CLOCK and NPAS2. The CRY proteins are expressed in both central and peripheral clock tissues.

DEC refers to Deleted in Esophageal Cancer (DEC) protein(s), also known as STRA13 and SHARP, which are members of the bHLH family and regulated in a circadian manner as well as responsive to hypoxia.

NUCLEAR HORMONE RECEPTOR refers to a family of transcriptional regulatory proteins that are activated by known and unknown ligands, including, but not limited to, glucocorticoids, thiazolidinediones, vitamin D3, thyroid hormone, estrogen, and androgens. The nuclear hormone receptors play critical roles in multiple metabolic functions, and members of the family show evidence of a circadian pattern of expression in peripheral clock tissues.

ADIPOSE TISSUE refers to the fat storing depots located throughout the body of immature and mature organisms, including but not limited to subcutaneous, omental, gonadal, interscapular, bone marrow, mammary, and mechanical sites.

ADULT STEM CELL refers to any undifferentiated cell found in a differentiated post-embryonic tissue that can renew itself and (with certain limitations) differentiate to yield the specialized cell types of the tissue from which it originated and into a wide variety of other cell types.

The term ADCs refers to adipose derived adult stem cells, isolated by collagenase digestion, and differential centrifugation from any adipose depot of mammalian or other vertebrate origin.

### MODES FOR CARRY OUT THE INVENTION

### Example 1

### Materials and Methods

All materials were obtained from Sigma/Aldrich (St. Louis, Mo.) or Fisher Scientific (Pittsburgh, Pa.) unless otherwise noted.

In Vivo Circadian Studies. Studies were conducted using 8-10 week old male AKR/J mice obtained from the Jackson Laboratories (Bar Harbor, Me.). The animals were acclimated to a regular chow diet (Purina 5015) ad libitum, under a strict 12-hr light: 12-hr dark cycle for 2 weeks. During this period, all animals were handled frequently by the staff to reduce the stress introduced by human contact. Following the acclimation period, animals were sacrificed in groups of 3 or 5 animals every 4 hr over a 48-hr period. Animals in the temporarily restricted feeding study were divided into a control cohort with ad libitum access to food and a Restricted feeding (RF) cohort with food access only during the 12-hr light period. Individual body weight and food intake were monitored daily for each animal during the 7-day restricted feeding period, and animals were killed in groups of 3 every 4 hr over a 24-hr period. Animals were killed by CO₂ asphyxiation and cervical dislocation, and harvested for serum, inguinal white adipose tissue (iWAT), epididymal WAT (eWAT), brown adipose tissue (BAT), and liver.

Quantitative Real-time RT-PCR (qRT-PCR) for Mouse Tissues. Total RNA was purified from tissues collected using TriReagent (Molecular Research Center) according to the manufacturer's specifications. Approximately 2 μg of total RNA was reverse transcribed using Moloney Murine Leukemia Virus Reverse Transcriptase (MMLV-RT; Promega), with Oligo dT at 42° C. for 1 hour in a 20 μL reaction. Primers for genes of interest were identified using Primer Express software (Applied Biosystems). A complete list of primers used in these studies is listed in Table 1. qRT-PCR was performed on diluted cDNA samples with SYBR® Green PCR Master Mix (Applied Biosystems) using the 7900 Real Time PCR system (Applied Biosystems) under universal cycling conditions (95° C. for 10 min; 40 cycles of 95° C. for 15 sec; then 60° C. for 1 min). All results were normalized relative to a Cyclophilin B expression control.

TABLE 1
Primers for Quantitative RT-PCR on Mouse Tissues

| | | |
|---|---|---|
| Mouse Gene | Forward Primer | Reverse Primer |

| | | |
|---|---|---|
| Arnt | TCGTTCATCTGCCGCATGA | TTCACAGAGCCAAGCCCATTC |
| Bmal1 | AACCTTCCCGCAGCTAACAG | AGTCCTCTTTGGGCCACCTT |
| Clock | GGCGTTGTTGATTGGACTAGG | GAATGGAGTCTCCAACACCCA |
| Cry1 | AGGAGGACAGATCCCAATGGA | GCAACCTTCTGGATGCCTTCT |
| Cry2 | AGCTGATGTGTTCCGAAGGCT | CATAATGGCTGCATCCCGTT |
| Cyclo B | GGTGGAGAGCACCAAGACAGA | GCCGGAGTCGACAATGATG |
| Dbp | GGAACTGAAGCCTCAACCAATC | CTCCGGCTCCAGTACTTCTCA |
| Npas2 | ACGCAGATGTTCGAGTGGAAA | CGCCCATGTCAAGTGCATT |
| Per1 | CCAGATTGGTGGAGGTTACTGAGT | GCGAGAGTCTTCTTGGAGCAGTAG |
| Per2 | AGAACGCGGATATGTTTGCTG | ATCTAAGCCGCTGCACACACT |
| Per3 | CCGCCCCTACAGTCAGAAAG | GCCCCACGTGCTTAAATCCT |
| Rev-erbα | CCCTGGACTCCAATAACAACACA | GCCATTGGAGCTGTCACTGTAG |
| Rev-erbβ | GGAACGGACCGTCACCTTT | TCCCCTGCTCCCATTGAGT |
| E4bp4 | AGAACCACGATAACCCATGAAAG | GACTTCAGCCTCTCATCCATCAA |
| Id2 | AGGCATCTGAATTCCCTTCTGA | AGTCCCCAAATGCCATTTATTTAG |
| Stra13 | GAGACGTGACGGGATTAACGA | CCAGAACCACTGCTTTTTCCA |

Serum Analysis. Commercially available ELISA kits for melatonin (Research Diagnostics, Flanders, N.J., Cat. # RE54021), leptin (Linco Research, Inc., St Louis, Mo., Cat. # EZML-82K), and radioimmunoassay kit for corticosterone (Mp Biomedicals, LLC, Orangeburg, N.Y., Cat. # 07-120102) were used according to the manufacturer's protocols. Assays were performed on serum samples pooled from n=3-5 animals harvested at individual time points. Corticosterone assays were performed in triplicate on pooled samples.

Periodicity Analysis. Periodicity of the circadian data obtained by qRT-PCR was tested with Time Series Analysis-Single Cosinor v. 6.0 software (Expert Soft Technologie). Each data set was fitted to a general cosine equation model:

A cos(2pt/T)+B sin(2pt/T)+M,

where A is the amplitude, T is the period (24 hours), and M is the MESOR (Midline Estimating Statistic Of Rhythm) (C. Bingham et al., 1982; W. Nelson et al., 1979). Model (ANOVA) was set as valid at the 0.950 probability level. The goodness of fit for each data set was tested with K-S (Kolomogorov and Smirnov), k², Average, and Q (Ljung-Box Q-statistic lack-of-fit hypothesis) tests; each individual data set reported has met acceptance criteria for each of these tests.

Affymetrix Oligonucleotide Microarray Gene Expression Analysis. RNA integrity was assessed by electrophoresis on the Agilent 2100 Bioanalyzer (Agilent Technologies, Palo Alto, Calif.). Double-stranded cDNA was synthesized from approximately 9 μg total RNA using a Superscript cDNA Synthesis Kit (Invitrogen, Carlsbad, Calif.) in combination with a T7-(dT)₂₄ primer. Biotinylated cRNA was transcribed in vitro using the GeneChip IVT Labeling Kit (Affymetrix, Santa Clara, Calif.) and purified using the GeneChip Sample Cleanup Module. Ten micrograms of purified cRNA was fragmented by incubation in fragmentation buffer (200 mM Tris-acetate, pH 8.1, 500 mM potassium acetate, 150 mM magnesium acetate) at 94° C. for 35 min and chilled on ice. Six and a half micrograms of fragmented biotin-labeled cRNA was hybridized to the Mouse Genome 430A 2.0 Array (Affymetrix), interrogating over 14,000 substantiated mouse genes. Arrays were incubated for 16 hr at 45° C. with constant rotation (60 rpm), washed, and then stained for 10 min at 25° C. with 10 μg/mL streptavidin-R phycoerythrin (Vector Laboratories, Burlingame, Calif.) followed by 3 μg/mL biotinylated goat anti-streptavidin antibody (Vector Laboratories) for 10 min at 25° C. Arrays were then stained once again with streptavidin-R phycoerythrin for 10 min at 25° C. After washing and staining, the arrays were scanned using a GeneChip Scanner 3000. Pixel intensities were measured, expression signals were analyzed, and features extracted using the commercial software package GeneChip Operating Software v.1.2 (Affymetrix). Data mining and statistical analyses were performed with Data Mining Tool v.3.0 (Affymetrix) algorithms. Arrays were globally scaled to a target intensity value of 2500 in order to compare individual experiments. The absolute call (present, marginal, absent) of each gene expression in each sample, and the direction of change, and fold change of gene expressions between samples were identified using the above-mentioned software.

Spectral Analysis of Microarray Data. Series of microarray expression values for gene x with N samples of the form x₀, x₁, x₂, . . . x_{N-1}, were converted from time-domain to a frequency domain using Discrete Fourier Transform (DFT) algorithm:

$$I ( ω ) = \frac{1}{N} {\vert {\sum }_{t = 0}^{N - 1} {x}_{t} {e}^{( - i ω t )} \vert}^{2} , ω ∈ [ 0 , π ]$$

Time series with a significant sinusoidal component with frequency ωε[0, π] showed a peak (periodogram) at that frequency with a high probability, unlike the purely random series whose periodogram approaches a flat line (M. B. Priestley, 1981). The significance of the observed periodicity was estimated by Fisher g-statistics, as recently recommended (S. Wichert et al., 2004). To account for multiple testing problems, the False Discovery Rate (FDR) method was used as a multiple comparison procedure (Y. Benjamini et al., 2001). This method is adaptive to the actual data and has been shown to control the FDR (S. Wickert et al., 2004; Y. Benjamini et al., 2001).

Circadian Gene Identification and Annotation. Circadian-expressed genes detected by Affymetrix microarray analysis were identified and annotated by matching the probe-set number with the gene information in the DAVID database.

Human Adipose-Derived Stem Cell (ASCs) Isolation and Expansion. All protocols were reviewed and approved by the Pennington Biomedical Research Center Institutional Research Board (IRB; Baton Rouge, La.) prior to the study. Liposuction aspirates from subcutaneous adipose tissue sites were obtained from female subjects undergoing elective procedures in local plastic surgical offices. Tissues were washed 3-4 times with phosphate-buffered saline (PBS) and suspended in an equal volume of PBS supplemented with 1% bovine serum and 0.1% collagenase type I (Worthington Biochemical Corporation, Lakewood, N.J.) prewarmed to 37° C. The tissue was then placed in a shaking water bath at 37° C. with continuous agitation for 60 min and centrifuged for 5 min at 300-500×g at room temperature. The supernatant, containing mature adipocytes, was aspirated. The pellet was identified as the stromal vascular fraction (SVF). Portions of the SVF were resuspended in cryopreservation medium (10% dimethylsulfoxide, 10% DMEM/F 12 Ham's, 80% fetal bovine serum), frozen at −80° C. in an ethanol-jacketed, closed container, and subsequently stored in liquid nitrogen. Portions of the SVF were used in colony forming unit assays (see below). The remaining cells of the SVF were suspended and plated immediately in T225 flasks in stromal medium (DMEM/F 12 Ham's, 10% fetal bovine serum (Hyclone, Logan, Utah), 100 U penicillin/100 μg streptomycin/0.25 μg Fungizone) at a density of 0.156 ml of tissue digest/sq cm of surface area for expansion and culture. This initial passage of the primary cell culture was referred to as “Passage 0” (P0). Following the first 48 hr of incubation at 37° C. at 5% CO₂, the cultures were washed with PBS and maintained in Stromal Media until they achieved 75-90% confluence (approximately 6 days in culture). The cells were passaged by trypsin (0.05%) digestion and plated at a density of 5,000 cells/cm² (“Passage 1”). Cell viability and numbers at the time of passage were determined by trypan blue exclusion and hemacytometer cell counts. Cells were passaged repeatedly after achieving a density of 75-90% (approximately 6 days in culture) until Passage 2.

Adipogenesis: Confluent cultures of primary adipose derived stem cells (Passage 2) were induced to undergo adipogenesis by replacing the stromal media with adipocyte induction medium composed of DMEM/F-12 with 3% FBS, 33 μM biotin, 17 μM pantothenate, 1 μM bovine insulin, 1 μM dexamethasone, 0.5 mM isobutylmethylxanthine (IBMX), 5 μM rosiglitazone, and 100 U penicillin/100 μg streptomycin/0.25 μg Fungizone, similar to the method described in U.S. Pat. No. 6,153,432. After three days, the medium was changed to adipocyte maintenance medium that was identical to induction media except for the deletion of both IBMX and rosiglitazone. Cells were maintained in culture for up to 9 days, with 90% of the maintenance media replaced every three days.

Circadian Induction: The medium was removed from confluent cultures of undifferentiated or adipocyte-differentiated human ASCs in 6 well plates and replaced with DMEM/Ham's F12 medium and 100 U penicillin/100 μg streptomycin/0.25 μg Fungizone alone or supplemented with one of the following: 30% FBS, 1 μM dexamethasone, or 5 μM rosiglitazone. The ASCs were exposed to the inductive agents for 2 hr. A single plate under each condition was harvested for total RNA after 1 hr of induction. After 2 hr, the medium in the remaining plates was replaced with serum free DMEM/Ham's F12 medium and 100 U penicillin/100 μg streptomycin/0.25 μg Fungizone alone. Individual plates were harvested for total RNA at 4 hr intervals up to 48 hr following the initial induction.

Quantitative Real-time RT-PCR (qRT-PCR) for Human ASCs: Total RNA was purified from the ASCs as described above for mouse tissues using TriReagent (Molecular Research Center) according to the manufacturer's specifications. Approximately 2 μg of total RNA was reverse transcribed using Moloney Murine Leukemia Virus Reverse Transcriptase (MMLV-RT; Promega), with Oligo dT at 42° C. for 1 hr in a 20 μL reaction. Primers for genes of interest (listed in Table 2) were identified using Primer Express software (Applied Biosystems). All primers were based on the sequence of the corresponding human mRNA and were designed to amplify across at least one exon/intron junction. qRT-PCR was performed on diluted cDNA samples with SYBR® Green PCR Master Mix (Applied Biosystems) using the 7900 Real Time PCR system (Applied Biosystems) under universal cycling conditions (95° C. for 10 min; 40 cycles of 95° C. for 15 sec; then 60° C. for 1 min). All results were normalized relative to a Cyclophilin B expression control.

TABLE 2
Primers for Quantitative RT-PCR on Human ASCs

| Accession # | Forward Primer | Reverse Primer |
|---|---|---|
| Gene Name | (Start site) | (Start site) |
| M60857 | GGAGATGGCACAGGAGGAAA | CGTAGTGCTTCAGTTTGAAGTTCTCA |
| Cyclophilin B | (316) | (388) |
| X72631 | GTTTGCCAAACACATCCCG | AAGCAAAGCGCACCATCAG |
| RevErb-a | (1558) | (1658) |
| NM_005126 | AACAAGCAAATCGAGTGCACC | TCCATAGTGGAATCCTGACGC |
| RevErb-b | (536) | (653) |
| NM_001178 | GTACCAACATGCAACGCAATG | TGTGTATGGATTGGTGGCACC |
| BMAL1 | (664) | (768) |
| NM_004898 | ACCCTTCCTCAACACCAACCA | ATGCGTGTCCGTTCCAA |
| CLOCK | (1678) | (1945) |
| NM_004075 | ATCTAGCCAGGCATGCAGTTG | GCTCCAATCTGCATCAAGCA |
| CRY1 | (1650) | (1759) |
| NM_021117 | CTGGATAAGCACTTGGAACGG | AGACAACCAAAGCGCAGGTAG |
| CRY2 | (731) | (849) |
| NM_002518 | TGGAGGCATTAGATGGCTTCA | ATCCGACGGTAAATGCCCA |
| NPAS2 | (555) | (655) |
| NM_002616 | ATTCCGCCTAACCCCGTATGT | GCCGCGTAGTGAAAATCCTCT |
| PER1 | (1132) | (1277) |
| NM_016831 | AGATGTCCTGGCGTCTTCTCA | TCATACCGTGCAGCTCTTTGG |
| PER3 | (652) | (780) |

Periodicity Analysis: Periodicity of the circadian data obtained by qRT-PCR was tested with Time Series Analysis-Single Cosinor v. 6.0 software (Expert Soft Technologie). Each data set was fitted to a general cosine equation model A cos(2pt/T)+B sin(2pt/T)+M, where A is the amplitude, T is the period (24 hours), and M is the MESOR (Midline Estimating Statistic Of Rhythm) (C. Bringham et al., 1982), providing the percentage of data points that behave in a rhythmic manner, and the r² value for the fit of the data set to the model curve. Model was also tested for validity at the 0.950 probability level (ANOVA). The goodness of fit for each data set was tested with K-S (Kolomogorov and Smirnov), k², Average, and Q (Ljung-Box Q-statistic lack-of-fit hypothesis) tests; each individual data set reported has met acceptance criteria for each of these tests.

### Example 2

### Adipose Tissues Express Circadian Oscillator Mechanism Genes

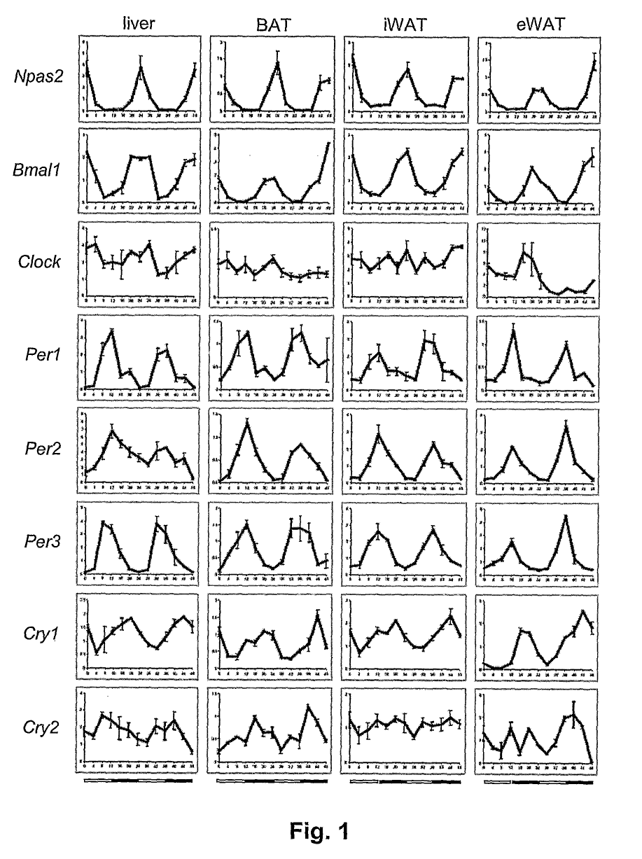

To investigate and characterize the presence of active peripheral circadian clocks in adipose tissues, a qRT-PCR approach was used to examine the circadian gene expression patterns in liver, and in brown, inguinal, and epididymal adipose tissues (BAT, iWAT, and eWAT respectively), of 8-week old AKR/J mice. Tissues were harvested from three male, 8-week-old AKR/J mice every 4 h over a 48-h period (13 time points, n=3 in each time point). Total RNA was extracted from collected tissues and used for quantitative RT-PCR analysis of gene expression, as described in Example 1. All values were normalized with the corresponding Cyclophilin B levels. In FIG. 1, all values are reported as averages±SD. As shown in FIG. 1, robust cyclic expression was found in the majority of circadian oscillator genes examined. Npas2 and Bmal1 cycled in synchrony, reaching their zenith (highest levels) around zeitgeber (circadian) time (ZT) 0 (0, 24, and 48 hr or the end of the 12-hr dark period), and their nadir (lowest levels) around ZT 12 (12 and 36 hr or the end of the 12-hr light period). Their expression patterns were consistent among BAT, iWAT, eWAT, and liver, with minor differences in the amplitudes. In contrast, Clock expression did not follow a consistent circadian pattern in any of these tissues (FIG. 1). Per1, Per2, and Per3 expression demonstrated synchronized 24-hr oscillations, reaching zenith around ZT 12 (12 and 36 hrs), and nadir around ZT 0 (0, 24, and 48 hrs) (FIG. 1). Although some inconsistencies were observed in the Cry2 expression, the overall gene expression of Cry1 and Cry2 followed a circadian profile, with a zenith around ZT 20 (20 and 44 hrs), and a nadir around ZT 8 (8 and 32 hrs) (FIG. 1). To confirm these findings the study was repeated several months later and the results displayed a close similarity to those in FIG. 1 (data not shown). The periodic nature of the observed gene expression patterns was demonstrated by fitting the expression data to cosine curves as mathematical models of periodic oscillatory patterns. (data not shown)

These results clearly demonstrate the presence of active peripheral circadian clocks in brown, inguinal, and epididymal adipose tissues. The robust cyclic expression of the circadian oscillator genes examined (Npas2, Bmal1, Per1-3, and Cry1-2) was consistent among BAT, iWAT, and eWAT, with minor differences in the amplitudes. Interestingly, the expression patterns mirrored those in liver, whose circadian clock has been independently characterized. (S. Panda et al., 2002b; K. A. Stokkan et al., 2001) However, the expression of Clock did not follow a consistent circadian pattern in any of these tissues, including liver. Others have shown that Clock expression, at least in the SCN, appears to be constitutive rather than cyclic. Also, in other peripheral tissues and forebrain, CLOCK protein actions were shown to be carried out by its orthologs, such as NPAS2 (M. Reick et al., 2001). The lack of an oscillatory expression profile for Clock does not exclude this gene as a component of the circadian oscillator in adipose tissue. The oscillatory pattern of Npas2 does not indicate that it is a critical component of the core oscillator in adipose tissue.

The slight lag observed between Cry and Per expression phase has been previously documented in the SCN and in certain peripheral tissues (N. R. Glossop et al., 2002). The daily oscillations of all Per and Cry genes examined occurred in a synchronous manner in BAT, iWAT, and eWAT, as well as in liver. Most importantly, in all tissues examined, the oscillations of Npas2 and Bmal1, occurred in anti-phase to those of Per and Cry, recapitulating the autoregulatory mechanisms of an active circadian clock, as identified in other mammalian tissues (R. Allada et al., 2001).

This conclusion was further supported by the results of the cosine-fit analysis, clearly showing that gene expression followed the harmonic trends of the cosine curve, as well as the anti-phase oscillations within the circadian clock. Together, these findings not only illustrate the presence of active circadian clock mechanisms in adipose tissues, but also confirm their periodic nature, in a consistent and reproducible manner.

### Example 3

### Circadian-Controlled Output Gene Oscillations in Adipose Tissues

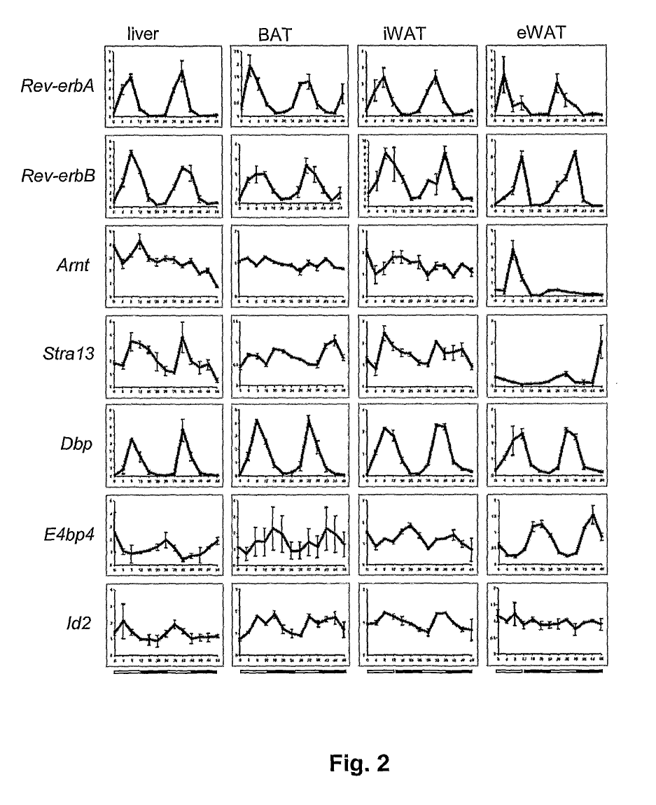

The presence of active circadian clocks in BAT, iWAT, and eWAT was further investigated by examining the expression levels of several genes known to be circadian-controlled in other tissues. The peripheral tissues of mice described above in Example 2 were used to determine the expression patterns for addition genes using qRT-PCR. As shown in FIG. 2, the expression of Rev-erbα and Rev-erbβ oscillated in phase with the Per genes in BAT, iWAT, and eWAT; and reflected the pattern observed in liver. The expression of Dbp showed an oscillatory pattern similar to Per and Rev-erb genes (FIG. 2), while the expression of E4 bp4 followed a circadian profile approximately in phase to Npas2 and Bmal1, and out of phase with Dbp (FIG. 2). Stra13 expression in fat tissues, especially in iWAT, showed a strong oscillatory trend, but did not follow a specific circadian pattern. Although Arnt gene expression did not fluctuate significantly, a circadian pattern of Id2 expression was observed in all tissues examined, including liver (FIG. 2).

The activity of peripheral circadian clocks is most evident through their effects on the expression patterns of several circadian-controlled output genes. REV-ERBα and REV-ERBβ are orphan nuclear hormone receptors that act as negative transcriptional regulators by binding ROR specific response elements (RORE) in gene promoters, thus preventing the binding of the positive transcription regulator RORα. They have also been shown to directly regulate the expression of Bmal1, Clock, and Cry1 through this mechanism (N. Preitner et al., 2002). The expression of Rev-erbα and Rev-erbβ is positively regulated by CLOCK:BMAL1, and negatively regulated by PER:CRY dimers, thus in agreement with the expression profile observed in this study. Expression of Rev-erbα has been shown to correlate with adipogenesis (A. Chawla et al., 1993), and its ectopic expression enhances adipocyte differentiation in vitro and in vivo (S. Laitinen et al., 2005). Thus, the circadian-regulated expression of these genes could play an important role in the adipocyte differentiation program.

DBP (albumin D-element binding protein) is a PAR-domain transcription factor, whose expression is under direct circadian control (L. Lopez-Molina et al., 1997). The expression of Dbp observed in this study is consistent with the findings that Dbp transcription can be driven by the CLOCK:BMAL1, and suppressed by PER:CRY, dimers (J. A. Ripperger et al., 2000). Furthermore, the studies in DBP-deficient mice have revealed that these animals exhibit altered activity periods, suggesting a role of Dbp in the control of circadian oscillatory mechanism (L. Lopez-Molina et al., 1997). DBP may also have a regulatory effect on the circadian oscillator mechanism since it has been shown to stimulate Per1 transcription (S. Yamaguchi et al., 2000).

The E4BP4 protein is closely related to DBP. Its promoter contains a RORE element, making it susceptible to transcriptional suppression by REV-ERB's (H. R. Ueda et al., 2002). Hence, although the E4 bp4 expression followed an oscillatory pattern, its phase was opposite to that of Dbp. (see also, S. Mitsui et al., 2001)

Stra13 (Dec1)) encodes a circadian-controlled transcriptional repressor/regulator of multiple genes, including several downstream circadian output genes A. Grechez-Cassiau et al., 2004). Stra13 transcription is activated by CLOCK:BMAL1, while the STRA13 protein acts as a repressor of CLOCK:BMAL1 activity (S. Honma et al., 2002). Maximal levels of Stra13 mRNA in liver have been reported to coincide with the peak of CLOCK:BMAL1 transcriptional activity (S. Panda et al., 2002b). Although a circadian oscillations was observed in liver, circadian expression of Stra13 was not observed in adipose tissues. This inconsistency may stem from the relatively low expression of Stra13 in these tissues.

ARNT is a bHLH-PAS domain protein, structurally similar to PER proteins (2). ARNT levels have been shown to follow a circadian oscillatory trend in liver, lung, and thymus, but not in spleen (V. M. Richardson et al., 1998). However, in the current work, no significant fluctuations of Arnt gene expression were seen in any of the adipose tissues or in liver. This may be indicative of diurnal changes in protein level through a post-translational, rather than a transcriptional control mechanism.

Id2 gene encodes a HLH protein lacking a DNA binding domain. ID2 proteins dimerize with other bHLH proteins, thereby inhibiting their DNA binding activities (K. Neuman et al., 1995). Its promoter contains E-box sequences making it a potential target for transcriptional regulation by circadian bHLH-PAS transcription factors. In a recent microarray analysis of SCN and liver, Id2 provided a prototype for a large cluster of circadian regulated genes (H. R. Ueda et al., 2002). Consistent with these findings, a strong oscillatory pattern was found in Id2 expression, with phase similar to that of other CLOCK-regulated genes studied, implying the involvement of the positive circadian regulators in Id2 expression.

### Example 4

### Serum Measures of Circadian Rhythm

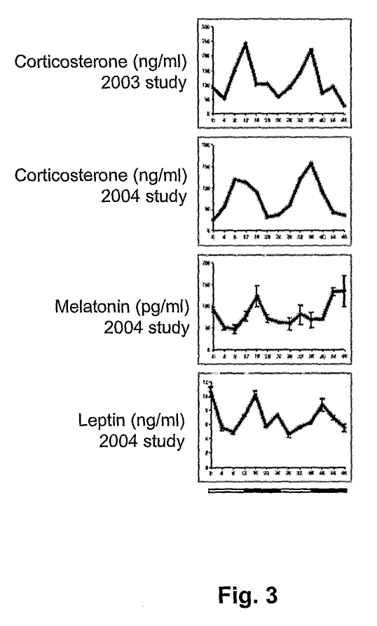

Blood samples were collected from male, 8-week-old, AKR/J mice every 4 hr over a 48-hr period (13 time points) in two independent studies. After centrifugation, sera from each time point were pooled and used for determination of serum proteins by radioimmunoassay (corticosterone) or ELISA (melatonin and leptin), as described in Example 1. All studies were performed on pooled samples (within a single time point). Values were reported in FIG. 3 as averages±SD. The serum corticosterone levels served as a systemic control and showed a circadian profile with zenith at the end of the 12-hr light period, similar to that of Per and Cry genes (FIG. 3). However, measurements of serum levels of melatonin and leptin yielded no significant oscillatory profiles.

Corticosterone levels are well known to display characteristic circadian rhythmicity (C. Allen et al., 1967), and were employed as controls. However, measurements of melatonin and leptin serum levels in cohorts of 5 animals showed trends of an oscillatory profile, but did not achieve significance. It is possible that significance would have been reached with a larger population base.

### Example 5

Microarray Analysis Reveals a Large Number of Periodically Expressed Genes in Adipose Tissues

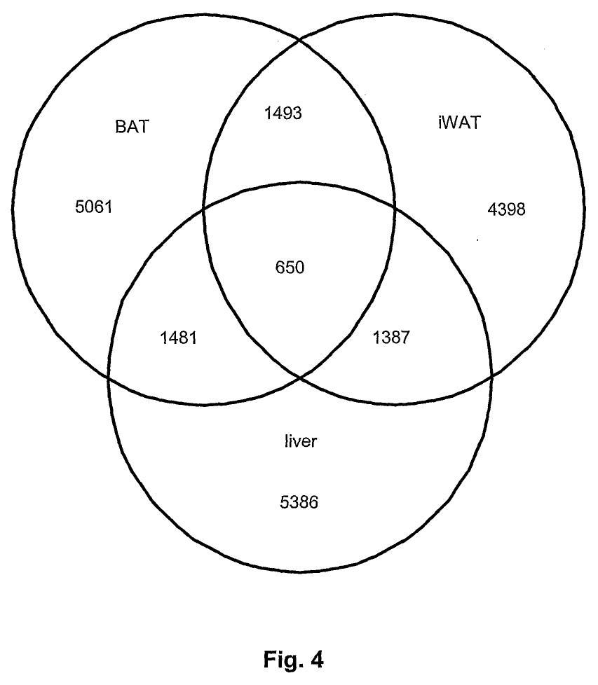

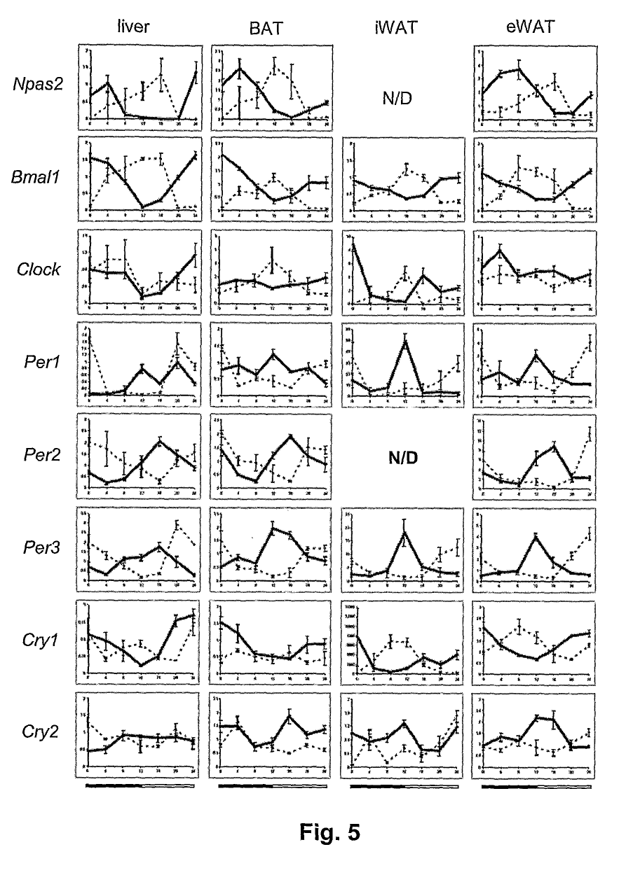

To determine the extent of circadian gene expression in adipose tissues, an Affymetrix microarray gene expression analysis was performed on the total RNA samples from the tissues previously examined by qRT-PCR, as described in Examples 1 and 2. After standard normalization, periodicity of gene expression was detected by discrete Fourier transformation, and significance of the circadian period was confirmed by Fisher's g-test, as described in Example 1. A large number of genes showed oscillatory expression patterns in iWAT (4398 genes), BAT (5061 genes), and liver (5386 genes). As shown in FIG. 4, 650 of these genes showed a conserved circadian expression pattern in BAT, iWAT, and liver, representing 14.8%, 12.8%, and 12% of the tissue specific-oscillatory transcriptome, respectively (FIG. 5). Although this group of genes was predominantly composed of those involved in basic metabolism and “house-keeping” functions, it also contained the circadian clock oscillator genes Npas2, Bmal1 (Arnt1), Per1, Per2, Per3, and Cry1, as well as the output gene Dbp, in agreement with our qRT-PCR studies (FIGS. 1 and 2) and confirmed by cosine-fit analysis (Data not shown). Furthermore, the expression of several genes involved in adipose function (Cebpα, Cebpγ, Lp1, Pparα, Pgc1β, and Stat5A) was also shown to cycle in these three tissues.

Another feature of the circadian transcriptome was that oscillating genes cycle in distinct temporal groups. Most of the genes could be grouped based on the zenith of their oscillatory phase, at either ZT 0, 4, 8, or 16, demonstrating that this pattern is detectable among shared oscillatory genes in BAT, iWAT, and liver. (Data not shown)

The results of the Affymetrix microarray gene expression analysis on the samples previously examined by qRT-PCR revealed a large number of genes with oscillatory expression patterns in iWAT, BAT, and liver. The detection of >5,300 genes exceeds previously reported values in the liver (R. A. Akhtar et al., 2002; K. Oishi et al., 2003). As described in Example 1, a frequency conversion approach was used to ensure that all periodically expressed genes were determined, regardless of their amplitude and “noise” level.

These findings suggest an overall coordination between the metabolic activities of these different tissues. This finding is further supported by the observation that oscillating genes cycle in distinct temporal groups, as evident among the oscillatory genes in BAT, iWAT, and liver.

### Example 6

### Temporally Restricted Feeding Regimen Alters the Circadian Expression Profile in Adipose Depots

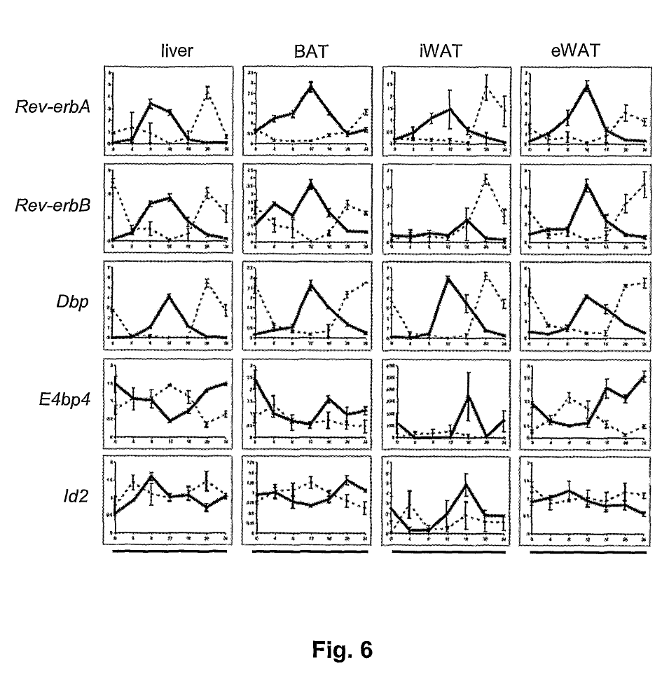

To determine whether the oscillatory patterns of gene expression in BAT, iWAT, and eWAT could be experimentally phase-shifted, food availability was temporally restricted to the 12-hr-light period in the experimental animal cohort (restricted feeding), whereas the control animal cohort are ad libitum. As further described in Example 1, forty-two male, 8-week-old, AKR/J mice were divided into two experimental groups, restricted feeding (solid line) and control (dashed line), and maintained in single housing for 7 days before they were killed. Liver, BAT, iWAT, and eWAT were harvested from three animals from each group every 4 hr over a 24-hr period (seven time points, two groups per time point, n=3 in each group per time point). Expression patters of candidate genes from liver, BAT, iWAT, and eWAT were determined and reported as described in Example 2. The results for several genes are shown in FIGS. 5 and 6. In FIG. 5, N/D means values were not determined. In this 24-hr study, as shown in FIGS. 5 and 6, the control animals (dashed lines) displayed circadian patterns of gene expression comparable with those seen in FIGS. 1 and 2. However, the animals whose food access was temporally restricted (solid lines) showed phase shifts in gene expression relative to control animals.

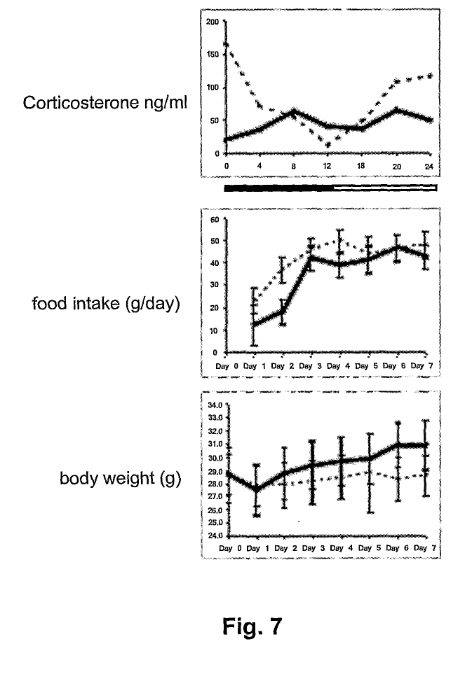

As additional controls, the serum cortisone, body weight, and food consumption were monitored during the 7-day temporal restricted feeding study. Blood samples were collected from animals as described above in Example 5. After centrifugation, sera from each group, within a given time point, were pooled and used for a single determination of serum corticosterone by radioimmunoassay. An individual animal's food intake and body weight were measured daily until killed and reported in FIG. 7 as averages±SD. As shown in FIG. 7, the temporally restricted feeding regimen led to a phase shift and amplitude dampening in the circadian pattern of corticosterone serum levels. There was no significant difference, past the initial adjustment period, in the food intake between the control and restricted feeding animals. Body weight was not significantly different between the groups, although a trend toward a body weight increase was observed in the restricted feeding groups.

Restricted feeding has been shown to regulate circadian rhythm in peripheral tissues in liver, skeletal muscle, heart, and kidneys (K. A. Stokkan et al., 2001; N. R. Glossop et al., 2002; K. Oishi et al., 2004; F. Damiola et al., 2000; N. Le Minh et al., 2001; and S. Yamazaki et al., 2000). In this current work, individual genes showed consistent phase shifts between liver, BAT, iWAT, and eWAT. However, the phase shifts observed were not uniform among all of the genes within individual tissues. Because the individual circadian clock components have unique regulatory mechanisms, restricted feeding may modulate each gene's phase differently. Similar observations have been made in liver (F. Damiola et al., 2000; N. Le Minh et al., 2001).

The antiphase relationship between the circadian oscillator genes was not affected by the temporal restricted feeding regiment. Instead, this relationship adjusted itself to the phase shifts of the individual genes, implying that these peripheral clocks possess mechanisms to adapt to entraining stimuli and varied physiological demands without compromising the oscillatory actions of the clock itself. Likewise, the temporal food restriction phase-shifted the output genes in a manner consistent with the oscillator genes regulating their expression, thereby recapitulating the connection between the oscillator function and output gene expression.

The current data indicate that restricted feeding also changes the oscillatory phase of serum corticosterone concentration, as previously reported (N. Le Minh et al., 2001).

### Example 7

### Effects of Dexamethasone on Circadian Gene Expression in Undifferentiated Human ASCs

Studies were conducted to examine the response of undifferentiated human ASCs to transient dexamethasone exposure. Passage 2 human ASCs were isolated from lipoaspirates obtained from healthy female donors undergoing elective surgery as described above. The donor ages ranged between 32 to 59 years while their BMIs ranged between 20.9 to 30.1 (Data not shown). Analysis of the ASCs from four of the donors in colony forming unit assays confirmed their ability to undergo both adipogenesis and osteogenesis in response to differentiation cocktails (as previously described in J. B. Mitchell et al., 2006). Initial studies were conducted with confluent and quiescent undifferentiated passage 2 ASCs obtained from these four donors. The ASCs were exposed either to serum free fresh medium alone or to medium supplemented with dexamethasone (1 μM) for a 2 hr period and then converted to serum free medium alone for up to 48 hr. Samples were harvested for total RNA an hour following induction and at 4 hr intervals following exposure initiation. The total RNA was used for qRT-PCR analysis of gene expression of Bmal1, Npas2, Cry1, Cry2, Per1, Per3, Rev-erbα, and Rev-erbβ, normalized relative to the corresponding Cyclophilin B levels.

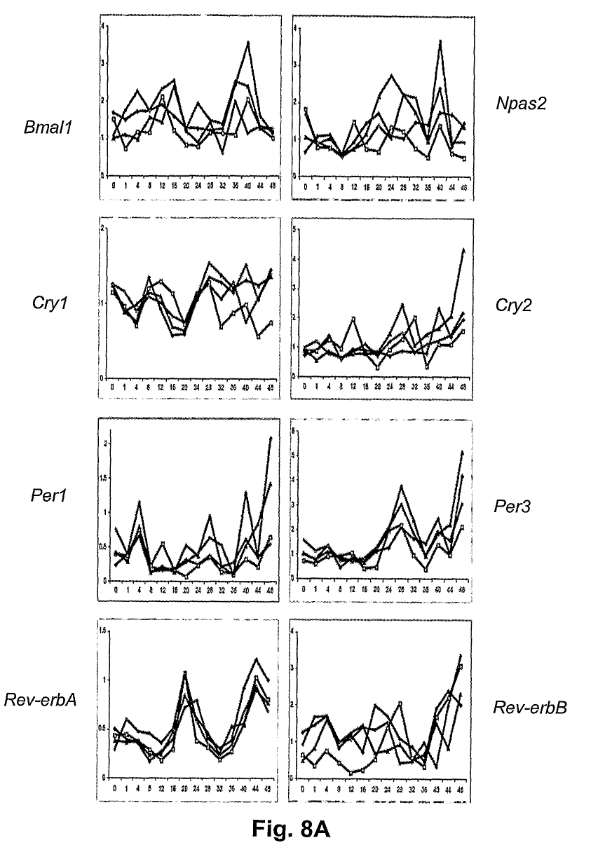

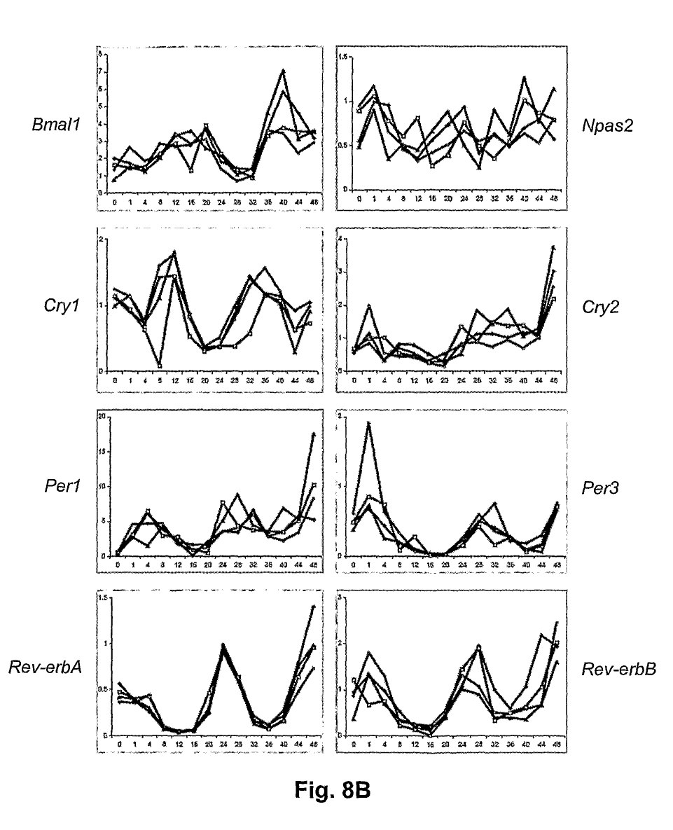

FIG. 8A shows the results of human ASCs exposed to fresh serum medium alone. Each line reflects results of cells from an individual donor. FIG. 8B shows the results of human ASCs exposed for 2 hr to dexamethasone and then planed in serum free medium. Exposure to fresh medium alone, containing glucose and other nutrients, induced a pronounced cyclic expression of Per3 and Rev-erbα in all subjects, with peak levels at 28 hours or 20 and 44 hr, respectively. The expression of the genes Bmal1, Cry1, Cry2, Npas2, and Per1 showed a trend towards an oscillatory profile; however, there was variability among the donors.

Following induction with dexamethasone, gene expression of Bmal1, Cry1, Cry2, Per1, Per3, and Rev-erbα exhibited an oscillatory profile in the undifferentiated ASCs from the four donors, as shown in FIG. 8B. Genes belonging to the “negative” regulatory arm of the circadian transcriptional apparatus, Per1, Per3, and Cry2, showed evidence of an immediate-early induction of mRNAs after 1 to 4 hr, followed by peaks between 28 to 32 hr and an elevation at 48 hr after induction. In contrast, Bmal1, a gene belonging to the “positive” regulatory arm of the circadian transcriptional apparatus, lacked an immediate-early induction and displayed peak levels 16 to 20 hr and 40 hr following induction. This reflects a phase shift of 8 to 12 hr relative to Per1, Per3, and Cry2. The gene encoding the CLOCK homolog and BMAL1 protein heterodimeric partner, NPAS2, did not display an oscillatory profile consistently among the four donors. The dexamethasone-induced expression profile of Rev-erbα was tightly regulated among the four donors, with peak levels at 24 hours and an elevation at 48 hours post induction.

### Example 8

### Induction of Circadian Gene Expression with Dexamethasone, Thiazolidinedione, or 30% Serum in Undifferentiated and Adipocyte-Differentiated Human ASCs

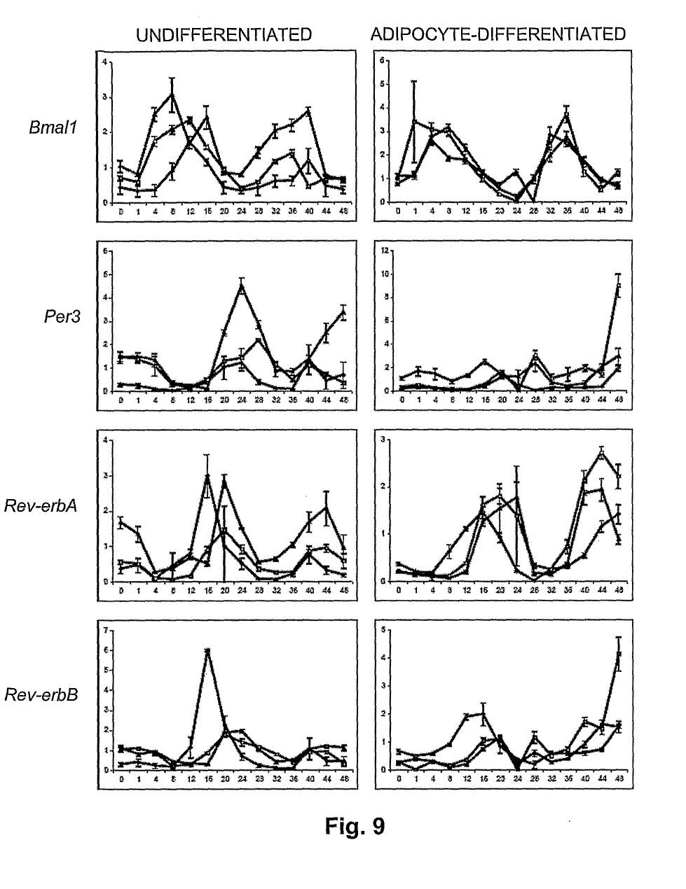

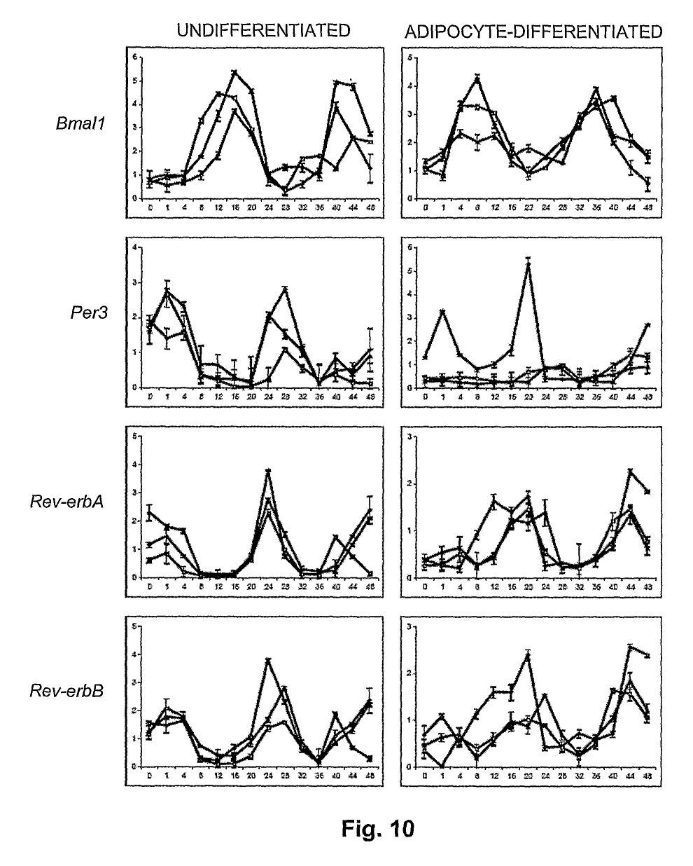

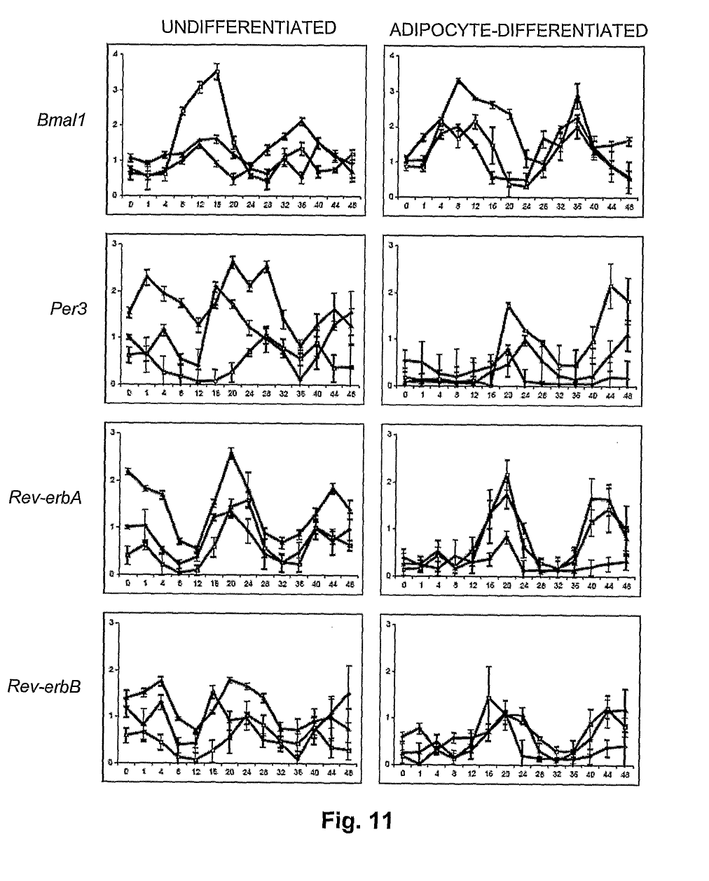

Since adipogenesis is accompanied by increased levels of the PPARγ2, further studies were conducted to determine whether adipocyte differentiated human ASCs responded differently to PPARγ2 or glucocorticoid receptor ligands. Serum shock (30% fetal bovine serum), known to induce circadian gene expression in rodent fibroblasts (6), was used as a control. Passage 2 human ASCs obtained from three individual donors were cultured to confluence and quiescence, as discussed above in Examples 1 and 7. Circadian gene expression was determined using ASCs in their undifferentiated state or after 9 days of adipocyte differentiation following exposure to an inductive cocktail containing dexamethasone or thiazolidinedione. The medium was removed from the undifferentiated and adipocyte differentiated cells and replaced with serum free medium supplemented with 30% fetal bovine serum (FIG. 9), dexamethasone (1 μM) (FIG. 10), or rosiglitazone (5 μM) (FIG. 11). The temporal expression profiles of Bmal1, Per 3, Rev-erbα, and Rev-erbβ normalized relative to Cyclophilin B were examined. The individual lines reflect values from the three different donors. The assays were performed in triplicate and values displayed are the mean±S.D.

The individual donors displayed variability in the amplitude of gene expression induced with the 3 different agents. In the undifferentiated and adipocyte-differentiated ASCs, each stimulus induced Bmal1 expression. The most rapid induction was achieved with 30% serum treatment, where the elevation and peak levels (zeniths) of gene expression occurred approximately 4 hr earlier (4-8 hr) relative to ones induced with dexamethasone and thiazolidinedione (8-12 hr). The period between successive peaks was ˜24-28 hr. The Per 3 responses to the individual stimuli varied. As noted in FIG. 8B, in undifferentiated ASCs, dexamethasone induced an immediate early response followed by a peak at 24-28 hr. The extent of the immediate early response was blunted in the adipocyte-differentiated ASCs where the subsequent peak occurred at 20-24 hrs. Neither 30% serum nor thiazolidinedione initiated an immediate early response, while the peak expression occurred at 24-28 hr in the undifferentiated ASCs and approximately 4 hr earlier in adipocyte-differentiated ASCs. The initiation of Rev-erbα and Rev-erbβ expression following the induction with dexamethasone or thiazolidinedione in undifferentiated ASCs was similar, reaching zenith at 24 hr and showing an elevation at 48 hr. In contrast, the undifferentiated ASC Rev-erbα and Rev-erbβ induction following a treatment with 30% serum in the undifferentiated ASCs was more rapid, reaching zenith at 12-20 hr. In the adipocyte-differentiated ASCs, all stimuli elicited peak induction of Rev-erbα and Rev-erbβ at 16-20 hr, with a second peak observed at 40-44 hr, corresponding to an ˜8 hr phase-advance for both dexamethasone and thiazolidinedione induction, relative to the undifferentiated ASCs.

The periodicity of the data was evaluated more rigorously using Cosinor analysis (Data not shown). The Cosinor “rhythm” determines the percentage of data points that behave in a rhythmic manner. For both undifferentiated and adipocyte differentiated ASCs, ≧50% of the data displayed statistically significant oscillatory rhythms. Two-thirds of the Bmal1, Rev-erbα, and Rev-erbβ datasets met ANOVA probability limits of 95%.

### Example 9

### Effects of GSK3B Inhibitor SB415286 on the Circadian Gene Expression Profiles of Human ASCs

Treatment of human adipose derived stem cells with dexamethasone was shown above to induce a circadian rhythm in vitro, and studies were conducted to see if this induction would be blocked by the addition of a glycogen synthase kinase 3 beta inhibitor. Confluent cultures of human ASCs were induced by exposure to 1 micromolar dexamethasone for a period of 2 hr. The medium was changed to either serum-free media (SF), or serum-free media with 30 μM SB415286 (SB415, an inhibitor of glycogen synthase kinase 3 beta). Individual cultures were harvested for total RNA at the indicated times and used for qRT-PCR analysis of gene expression of Bmal1, Per3, and Rev-erbα, and normalized relative to the corresponding Cyclophilin B levels. Assays were performed in triplicate and values displayed are the mean±S.D.

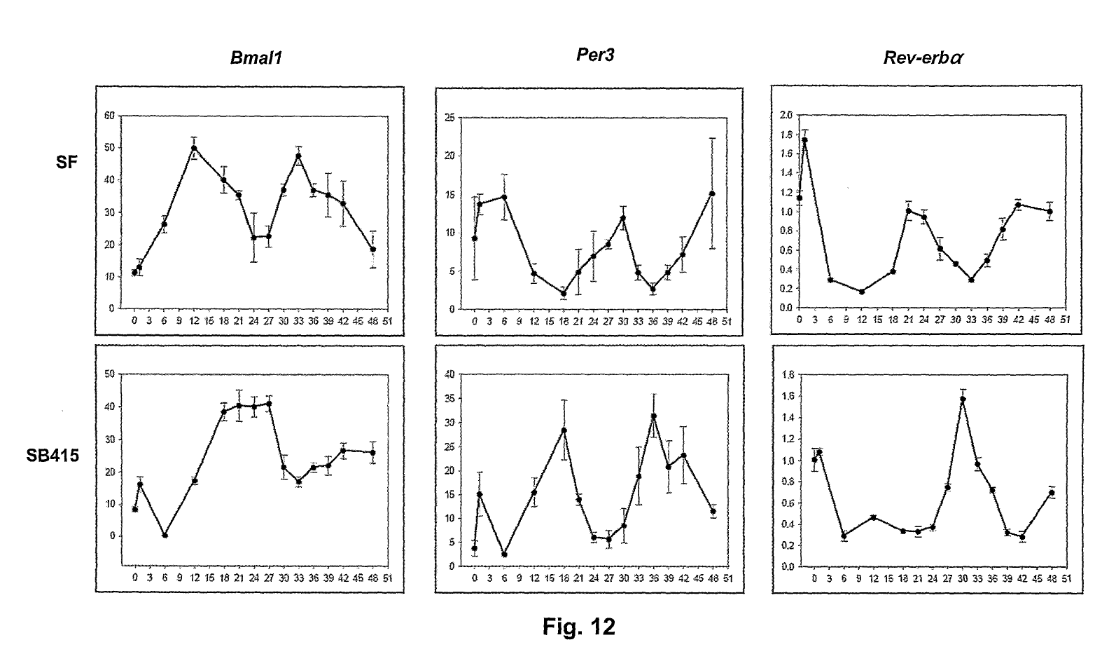

As shown in FIG. 12, the circadian cycle was shifted and lengthened when cells were incubated with SB415. In the absence of the SB415286, the dexamethasone induced cells displayed a time dependent increase in the expression of the mRNA levels of the circadian transcription factor genes BMAL1 and Per 3, as well as the downstream targets, Rev-erb alpha and Rev-erb beta. (Data for Rev-erb beta not shown) In the presence of the glycogen synthase kinase 3 beta inhibitor, the mRNA induction profile was delayed and shifted by 6-9 hours, consistent with a lengthening of the circadian cycle or tau. These results indicate that the circadian cycle of gene expression in adipose cells can be shifted by using an inhibitor of glycogen synthase kinase 3 beta. Other known inhibitors of glycogen synthase kinase 3 beta include SB-216763, lithium, insulin, and phenylephrine (M. P. Coghlan et al., 2000; D. A. Cross et al., 2001; K. MacAulay et al., 2003; and L. M. Ballou et al., 2001).

### Example 10

### Induction of Weight Gain by Lengthening the Circadian Day with SB415286

Treatment of mice with a glycogen synthase kinase 3 beta inhibitor (SB415286) will induce obesity by lengthening the mean circadian day (tau). Studies will be performed in AKR/J mice. Cohorts will be placed on a regular chow diet and maintained under a constant 12 hr light: 12 hr dark (LD) or constant dark (DD) photic cycle. Animals will be treated with a placebo or with a GSKbeta inhibitor (SB415286) daily by gavage for a period of 4 to 8 weeks. Animal weights will be monitored daily. Wheel running will be monitored continuously, as a surrogate marker for activity and circadian rhythms. Treatment with the GSK 3 beta inhibitor will cause the daily circadian cycle to increase by 3% to 10% daily under constant dark conditions (DD) relative to controls. Statistically significant increases in animal body weight will occur between the control and GSK 3 beta inhibitor cohorts under DD conditions by the end of the 8 week period.

In a second experiment, mice will be placed under the identical conditions except that they will receive a high fat diet (45% calories from fat). Treatment with the GSK3beta inhibitor will cause a significant elevation in body weight relative to the placebo control group by the end of the 8 week period. Animals will be sacrificed periodically to obtain RNA from the adipose tissues and measure the gene expression of the peripheral clock genes as a function of time. The addition of the GSK3 beta inhibitor will correlate with a phase shift in the circadian core transcriptional apparatus mRNA expression profile in adipose tissue depots and liver in the experimental group relative to the controls.

### Example 11

### Induction of Weight Gain by Lengthening the Circadian Period with Lithium Chloride

Treatment of mice with another glycogen synthase kinase 3 beta inhibitor (lithium chloride) will induce obesity and lengthen the mean circadian day (tau). Studies will be performed in AKR/J mice. Cohorts will be placed on a regular chow diet and maintained under a constant 12 hr light: 12 hr dark (LD) or constant dark (DD) photic cycle. Animals will receive regular drinking water (Control) or drinking water containing lithium chloride (concentration) for a period of 4 to 8 weeks. Animal weights will be monitored daily. Wheel running will be monitored continuously as a surrogate marker for activity and circadian rhythms. Treatment with lithium chloride will cause the daily circadian cycle to increase by 3% to 10% daily under constant dark conditions (DD) relative to controls. Statistically significant increases in animal body weight will occur between the control and lithium chloride cohorts under DD conditions by the end of the 8 week period. Treatment with the lithium chloride will cause a significant elevation in body weight relative to the placebo control group by the end of the 8 week period. Animals will be sacrificed periodically to obtain RNA from the adipose tissues and measure the gene expression of the peripheral clock genes as a function of time. The addition of lithium chloride will correlate with a phase shift in the circadian core transcriptional apparatus mRNA expression profile in adipose tissue depots and liver in the experimental group relative to the controls.

### Example 12

### Induction of Weight Gain by Lengthening the Circadian Period with an Agent Used to Treat Bipolar Disorder, Valproic Acid

Treatment of mice with agents used to treat bipolar disorder will induce obesity and lengthen the circadian day (tau). Studies will be performed in AKR/J mice. Cohorts will be placed on a regular chow diet and maintained under a constant 12 hr light: 12 hr dark (LD) or constant dark (DD) photic cycle. Animals will receive a placebo (Control) or valproic acid (concentration) by oral gavage daily for a period of 4 to 8 weeks. Animal weights will be monitored daily. Wheel running will be monitored continuously as a surrogate marker for activity and circadian rhythms. Treatment with the valproic acid or other anti-bipolar disorder agent will cause the daily circadian cycle to increase by 3% to 10% daily under constant dark conditions (DD) relative to controls. Statistically significant increases in animal body weight will occur between the control and valproic acid cohorts under DD conditions by the end of the 8 week period. Treatment with the bipolar treatment compound will cause a significant elevation in body weight relative to the placebo control group by the end of the 8 week period. Animals will be sacrificed periodically to obtain RNA from the adipose tissues and measure the gene expression of the peripheral clock genes as a function of time. The addition of valproic acid will correlate with a phase shift in the circadian core transcriptional apparatus mRNA expression profile in adipose tissue depots in the experimental group relative to the controls.

In a second experiment, mice will be placed under the identical conditions except that they will receive a high fat diet (45% calories from fat). Treatment with the GSK3beta inhibitor will cause a significant elevation in body weight relative to the placebo control group by the end of the 8 week period. This will correlate with a phase shift in the circadian core transcriptional apparatus mRNA expression profile in adipose tissue depots and liver in the experimental group relative to the controls.

### Example 13

### Induction of Weight Loss by Shortening the Circadian Period with a GSK 3 Beta Activator

Treatment of mice with a glycogen synthase kinase 3 beta activator will reduce obesity and shorten the mean circadian day (tau). Studies will be performed in AKR/J mice. Cohorts will be placed on a regular chow diet and maintained under a constant 12 hr light: 12 hr dark (LD) or constant dark (DD) photic cycle. Animals will receive a placebo (Control) or a glycogen synthase kinase 3 beta activator (concentration) by oral gavage daily for a period of 4 to 8 weeks. Animal weights will be monitored daily. Wheel running will be monitored continuously as a surrogate marker for activity and circadian rhythms. Treatment with the GSK 3 beta activator will cause the daily circadian cycle to decrease by 3% to 10% daily under constant dark conditions (DD) relative to controls. Statistically significant decreases in animal body weight will occur between the control and experimental treatment cohorts under DD conditions by the end of the 8 week period. Treatment with the GSK30 activator will cause a significant elevation in body weight relative to the placebo control group by the end of the 8 week period. Animals will be sacrificed periodically to obtain RNA from the adipose tissues and measure the gene expression of the peripheral clock genes as a function of time. The addition of the GSK3beta activator will correlate with a phase shift in the circadian core transcriptional apparatus mRNA expression profile in adipose tissue depots and liver in the experimental group relative to the controls. Examples of known GSK3beta activators include octreotide (a somatostatin analog), somatostatin, enzastaurin, and aspirin (M. Theodoropoulou et al., 2006; J. R. Graff et al., 2005; and A. di Palma et al., 2006).

In a second experiment, mice will be placed under the identical conditions except that they will receive a high fat diet (45% calories from fat). Treatment with the GSK3beta activator will cause a significant reduction in body weight relative to the placebo control group by the end of the 8 week period. Animals will be sacrificed periodically to obtain RNA from the adipose tissues and measure the gene expression of the peripheral clock genes as a function of time. Again, the addition of the GSK3beta activator will correlate with a phase shift in the circadian core transcriptional apparatus mRNA expression profile in adipose tissue depots and liver in the experimental group relative to the controls.

### Example 14

### A High Fat Diet Alters the Peripheral Circadian Clock Gene Apparatus in Adipose Tissue

Studies will be conducted to confirm that treatment with a high fat diet alters the circadian expression profile of the “clock” gene family in adipose tissues from multiple depots. Male AKR/J mice (age 6-8 weeks) will be purchased from the Jackson Laboratory and housed in the Pennington Biomedical Research Center Comparative Animal Facility for a period of 2 weeks under a strictly maintained 12 hr light/12 hr dark cycle. Cohorts of 32-40 animals will be maintained on an ad lib regular chow diet or on a high fat diet (20-30%) for 2 weeks. Groups of 3-4 animals from each cohort will be euthanized by cervical dislocation at 3 to 4 hr intervals for up to a 36 to 48 hr period. The following tissues will be harvested: serum, adipose tissues (subcutaneous, epididymal, omental, interscapular, retroperitoneal), heart, liver, skeletal muscle, bone/cartilage). Tissue samples will be flash frozen and subsequently harvested for total RNA and protein. Serum samples will be analyzed for expression of adiponectin, leptin, and/or agouti-related protein by ELISA assay. Samples will be analyzed for protein by Western immunoblot and for RNA by real time PCR and/or Northern blot analysis for expression of the “clock” genes, including but not limited to some or all of the following genes: Cry 1, Cry 2, Per 1, Per 2, Per 3, Clock, BMAL1, NPAS2, DEC1, DEC2, Rev-Erbα, Rev-Erbβ. Appropriate controls (actin, cyclophilin and/or GAPDH) will be performed in parallel. In addition, adipose tissue expression of adiponectin, leptin, lipoprotein lipase, PPAR gamma and PPAR alpha will be determined. Control studies will examine expression profile of the same gene products in liver tissue. Other tissues will be stored for further analysis. Evidence of the induction profile of the gene products will be determined and analyzed mathematically for evidence of an oscillatory pattern as shown above.

### Example 15

### Human Subjects have an In Vivo Peripheral Circadian Clock in Adipose Tissue

Studies will be conducted to confirm that human subjects express an in vivo circadian clock apparatus in subcutaneous adipose tissues. Obese (BMI>30) subjects will be recruited to the study (10<n<20). Subjects will be maintained on a strict 12 hr light/12 hr dark cycle and ad lib diet for 7 days. Over the final 36 hours, the patients will have an indwelling catheter placed in an appropriate venous access site. Blood samples will be harvested for serum at 20-30 min intervals. At 3 hr intervals, the patients will undergo needle biopsies of subcutaneous adipose depots on the thigh, upper arm, abdomen, and buttocks in a randomized, pre-programmed harvest pattern. Tissue samples will be flash frozen and subsequently harvested for total RNA. Serum samples will be analyzed for expression of adiponectin, leptin, and/or agouti related protein by ELISA assay. Tissue samples will be analyzed for RNA by real time PCR analysis for expression of the “clock” genes, including but not limited to some or all of the following products: Cry 1, Cry 2, Per 1, Per 2, Per 3, Clock, BMAL1, NPAS2, DEC1, DEC2, rev-Erbα, rev-Erbβ. Appropriate controls (actin, cyclophilin and/or GAPDH) will be performed in parallel. In addition, adipose tissue expression of adiponectin, leptin, lipoprotein lipase, PPAR gamma and PPAR alpha will be determined. The expression profile of the gene products will be determined and analyzed mathematically for evidence of an oscillatory pattern.

### Example 16

### Peripheral Circadian Clock Gene Apparatus in Adipose Tissue of Human Subjects Can be Entrained

Obese (BMI>30) subjects will be recruited to the study (20≦n≦40). Subjects will be maintained on a strict 12 hr light/12 hr dark cycle 14 days. Half of the subjects will receive a routine, ad lib diet during this period at routine intervals during the 12 hr light cycle. An age and gender matched cohort will receive the same diet at the same intervals during the 12 hr dark cycle. Over the final 36 hours, the patients will have an indwelling catheter placed in an appropriate venous access site. Blood samples will be harvested for serum at 20-30 min intervals. At 3 hr intervals, the patients will undergo needle biopsies of subcutaneous adipose depots on the thigh, upper arm, abdomen, and buttocks in a randomized, pre-programmed harvest pattern. Tissue samples will be flash frozen and subsequently harvested for total RNA. Serum samples will be analyzed for expression of glucocorticoid by RNA and for adiponectin, leptin, and/or agouti related protein by ELISA assay. Tissue samples will be analyzed for RNA by real time PCR analysis for expression of the “clock” genes, including but not limited to some or all of the following products: Cry 1, Cry 2, Per 1, Per 2, Per 3, Clock, BMAL1, NPAS2, DEC1, DEC2, rev-Erbα, rev-Erbβ. Appropriate controls (actin, cyclophilin and/or GAPDH) will be performed in parallel. In addition, adipose tissue expression of adiponectin, leptin, lipoprotein lipase, PPAR gamma and PPAR alpha will be determined. The expression profile of the gene products will be determined and analyzed mathematically for evidence of an oscillatory pattern. The two cohorts will be compared with respect to the expression profile of the clock genes and other adipose tissue products with respect to the circadian rhythm].

I have shown the presence of an active peripheral circadian clock in adipose tissue depots which suggest that there is a temporal component to the regulation of adipose tissue function. Other evidence linking circadian dysfunction to obesity and the metabolic syndrome also support this idea. I have also shown that human ASCs provide a robust in vitro model for temporal molecular analyses of subcutaneous adipose tissue. The transient exposure of ASCs to serum shock or to a nuclear hormone receptor ligand induced expression of genes involved in the core circadian transcriptional apparatus (Bmal1, Per, Cry) and their immediate effectors (Rev-erbα & β). In general, serum shock induced cyclic gene expression ˜4 hr in advance of an equivalent exposure to nuclear hormone receptor ligands. Moreover, the response to nuclear hormone receptor ligands by adipocyte-differentiated ASCs preceded that of undifferentiated ASCs by ˜4-8 hr. However, since both dexamethasone and rosiglitazone have been used to initiate adipogenesis in the ASCs, the differentiated cells may be sensitized to nuclear hormone receptor ligands. In addition, the levels of thiazolidinedione receptor, PPARγ2, are known to increase during human ASC adipocyte differentiation (Y. D. Halvorsen et al., 2001). Either or both of these facts could account for accelerated nuclear hormone receptor ligand response of the adipocyte-differentiated ASCs relative to their undifferentiated counterparts.

These results further confirm that circadian mechanisms can vary in cells isolated from individual donors. I have documented for the first time the oscillation of core circadian transcriptional apparatus in adipose tissue depots. Exogenous stimuli, such as temporal restriction to food access, was found to phase-shift this expression profile. I have also found that certain GSK3 beta inhibitors can be used to lengthen the circadian expression profile, and could be used to induce weight gain in patients suffering from cachexia. I have also shown that isolated ASCs have the potential to serve as a surrogate in vitro model for analysis of circadian mechanisms in human adipose tissue. The temporal kinetics of circadian gene induction in human ASCs changed as a function of their differentiation status. Thus, mature adipocytes may differ from preadipocytes with respect to their response to nuclear hormone receptor ligands in vivo, whether these are endogenous hormones, such as corticosterone, or exogenous medications, such as oral anti-diabetic agents. Consistent with chronobiological models, the time of day when thiazolidinediones are administered to diabetic patients may have a significant impact on their therapeutic effects due to the circadian clock in the adipose tissue.

### Miscellaneous

The term “therapeutically effective amount” as used herein refers to an amount of a compound to lengthen or shorten the circadian pattern of gene expression of certain clock genes found in adipose tissue to a statistically significant degree (p<0.05). The term “therapeutically effective amount” therefore includes, for example, an amount sufficient to lengthen the pattern of gene expression sufficient to cause weight gain in a patient in such need of weight, for example, one suffering from cachexia or anorexia nervosa. The dosage ranges for the administration of the compound are those that produce the desired effect. Generally, the dosage will vary with the form of delivery. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the extent of circadian pattern changes and of weight change by methods well known to those in the field. The application of the compound can be oral, by injection, or topical, but with the compound targeted to adipose tissue by making the compound or its carrier lipophilic. In particular, the compounds could be delivered transdermally directly over subcutaneous adipose tissue in the form of a lipophilic cream or a slow-release subcutaneous implant. Alternatively the compound could be injected directly into the subcutaneous adipose tissue.

### LITERATURE

Akhtar, R. A. et al., “Circadian cycling of the mouse liver transcriptome, as revealed by cDNA microarray, is driven by the suprachiasmatic nucleus,” Curr Biol., Vol. 12:, pp. 540-550 (2002)
Allada, R. et al., “Stopping time: the genetics of fly and mouse circadian clocks,” Annu Rev Neurosci, Vol. 24, pp. 1091-1119 (2001)
Allen, C. et al., “Maturation of the circadian rhythm of plasma corticosterone in the rat,” Endocrinology, Vol. 80, pp. 926-930 (1967)
Ando, H. et al., “Rhythmic mRNA Expression of Clock Genes and Adipocytokines in Mouse Visceral Adipose Tissue,” Endocrinology (2005)
Arvidson, N. G. et al., “The timing of glucocorticoid administration in rheumatoid arthritis,” Ann Rheum Dis., Vol. 56, no. 1, pp. 27-31 (January 1997)
Balsalobre, A. et al., “A serum shock induces circadian gene expression in mammalian tissue culture cells,” Cell, Vol. 93, pp. 929-937 (1998)
Balsalobre, A. et al., “Resetting of circadian time in peripheral tissues by glucocorticoid signaling,” Science, Vol. 289, no. 5488, pp. 2344-2347 (2000)
Balsalobre, A. et al., “Multiple signaling pathways elicit circadian gene expression in cultured Rat-1 fibroblasts,” Curr Biol, Vol. 10, no. 20, pp. 1291-1294 (2000)
Benavides, A. et al., “Circadian rhythms of lipoprotein lipase and hepatic lipase activities in intermediate metabolism of adult rat,” Am J Physiol, Vol. 275, pp. R811-817 (1998)
Benjamini, Y. et al., “Controlling the false discovery rate in behavior genetics research,” Behav Brain Res, Vol. 125, pp. 279-284 (2001)
Bingham, C. et al., “Inferential statistical methods for estimating and comparing cosinor parameters,” Chronobiologia, Vol. 9, pp. 397-439 (1982)
Birketvedt, G. S. et al., “Behavioral and neuroendocrine characteristics of the night-eating syndrome,” Jama, Vol. 282, pp. 657-663 (1999)
Brown, S. A. et al., “Rhythms of mammalian body temperature can sustain peripheral circadian clocks,” Curr Biol, Vol. 12, pp. 1574-1583 (2002)
Brown, S. A. et al., “The period length of fibroblast circadian gene expression varies widely among human individuals,” PLoS Biol, Vol. 3, p. e338 (2005)
Chawla, A. et al., “Induction of Rev-ErbA alpha, an orphan receptor encoded on the opposite strand of the alpha-thyroid hormone receptor gene, during adipocyte differentiation,” J Biol. Chem., Vol. 268, no. 22, pp. 16265-9 (Aug. 5, 1993)
Chopin-Delannoy, S. et al., “A specific and unusual nuclear localization signal in the DNA binding domain of the Rev-erb orphan receptors,” J Mol. Endocrinol., Vol. 30, no. 2, pp. 197-211 (April 2003)
Coghlan, M. P. et al., “Selective small molecule inhibitors of glycogen synthase kinase-3 modulate glycogen metabolism and gene transcription,” Chem Biol, Vol. 7, pp. 793-803 (2000).
Cross, D. A. et al., “Selective small-molecule inhibitors of glycogen synthase kinase-3 activity protect primary neurons from death,” J Neurochem, Vol. 77, pp. 94-102 (2001).
Cutolo, M. et al., “Circadian rhythms in RA,” Ann Rheum Dis., Vol. 62, no. 7, pp. 593-6 (July 2003)
Czeisler, C. A. et al., “Stability, precision, and near-24-hour period of the human circadian pacemaker,” Science, Vol. 284, no. 5423, pp. 2177-81 (Jun. 25, 1999)
Damiola, F. et al., “Restricted feeding uncouples circadian oscillators in peripheral tissues from the central pacemaker in the suprachiasmatic nucleus,” Genes Dev., Vol. 14, no. 23, (Dec. 1, 2000)
Darlington, T. K. et al., “Closing the circadian loop: CLOCK-induced transcription of its own inhibitors per and tim,” Science, Vol. 280, pp. 1599-1603 (1998)
Delany, J. et al., “Proteomic analysis of primary cultures of human adipose derived stem cells: Modulation by adipogenesis,” Mol Cell Proteomics, Vol. 4, pp. 731-740 (2005)
Di Palma, A. et al., “Aspirin reduces the outcome of anticancer therapy in Meth A-bearing mice through activation of AKT-glycogen synthase kinase signaling,” Mol Cancer Ther, Vol. 5, pp. 1318-24 (2006).
Duffield, G. E. et al., “Circadian programs of transcriptional activation, signaling, and protein turnover revealed by microarray analysis of mammalian cells,” Curr Biol, Vol. 12, pp. 551-557 (2002)
Durgan, D. J. et al., “The intrinsic circadian clock within the cardiomyocyte,” Am J Physiol Heart Circ Physiol, Vol. 289, pp. H1530-1541 (2005)
Elimam, A. et al., “Variations in glucocorticoid levels within the physiological range affect plasma leptin levels,” Eur J. Endocrinol., Vol. 139, no. 6, 615-20 (December 1998)
Fagiolini, A. et al., “Prevalence of obesity and weight change during treatment in patients with bipolar I disorder,” J Clin Psychiatry, Vol. 63, pp. 528-533 (2002)
Fagiolini, A. et al., “Obesity as a correlate of outcome in patients with bipolar I disorder,” Am J Psychiatry, Vol. 160, pp. 112-117 (2003)
Gavrila, A. et al., “Diurnal and ultradian dynamics of serum adiponectin in healthy men: comparison with leptin, circulating soluble leptin receptor, and cortisol patterns,” J Clin Endocrinol Metab, Vol. 88, pp. 2838-2843 (2003)
Gavrila, A. et al., “Serum adiponectin levels are inversely associated with overall and central fat distribution but are not directly regulated by acute fasting or leptin administration in humans: cross-sectional and interventional studies,” J Clin Endocrinol Metab, Vol. 88, pp. 4823-4831 (2003)
Gekakis, N. et al., “Role of the CLOCK protein in the mammalian circadian mechanism,” Science, Vol. 280, pp. 1564-1569 (1998)
Gimble, J. M. et al., “Differentiation potential of adipose derived adult stem (ADAS) cells,” Curr Top Dev Biol, Vol. 58, pp. 137-160 (2003)
Glossop, N. R. et al., “Central and peripheral circadian oscillator mechanisms in flies and mammals,” J Cell Sci, Vol. 115, pp. 3369-3377 (2002)
Graff, J. R. et al., “The protein kinase Cbeta-selective inhibitor, Enzastaurin (LY317615.HCL), suppresses signaling through the AKT pathway, induced apoptosis, and suppresses growth of human colon cancer and glioblastoma xenografts,” Cancer Res., Vol. 65, pp. 7462-9 (2005).
Grechez-Cassiau, A. et al., “The transcriptional repressor STRA13 regulates a subset of peripheral circadian outputs,” J Biol Chem, Vol. 279, pp. 1141-1150 (2004)
Griffin, E. A. Jr., et al., “Light-independent role of CRY1 and CRY2 in the mammalian circadian clock,” Science, Vol. 286, pp. 768-771 (1999)
Halvorsen, Y. D. et al., “Thiazolidinediones and glucocorticoids synergistically induce differentiation of human adipose tissue stromal cells: biochemical, cellular, and molecular analysis,” Metabolism, Vol. 50, pp. 407-413 (2001)
Holmback, U. et al., “Endocrine responses to nocturnal eating—possible implications for night work,” Eur J Nutr, Vol. 42, pp. 75-83 (2003)
Honma, S. et al., “Dec1 and Dec2 are regulators of the mammalian molecular clock,” Nature, Vol. 419, no. 6909, pp. 841-844 (October 2002)
la Fleur, S. E. et al., “A daily rhythm in glucose tolerance: a role for the suprachiasmatic nucleus,” Diabetes, Vol. 50, pp. 1237-1243 (2001)
Laitinen, S. et al., “The role of the orphan nuclear receptor Rev-Erb alpha in adipocyte differentiation and function,” Biochimie, Vol. 87, pp. 21-25 (2005)
Le Minh, N. et al., “Glucocorticoid hormones inhibit food-induced phase-shifting of peripheral circadian oscillators,” EMBO J., Vol. 20, no. 24, pp. 7128-36 (December 2001)
Lemberger, T. et al., “Expression of the peroxisome proliferator-activated receptor alpha gene is stimulated by stress and follows a diurnal rhythm,” J Biol. Chem., Vol. 27, no. 3, pp. 1764-9 (January 1996)
Lopez-Molina, L. et al., “The DBP gene is expressed according to a circadian rhythm in the suprachiasmatic nucleus and influences circadian behavior,” Embo J, Vol. 16, pp. 6762-6771 (1997)
Lowrey, P. L. et al., “Mammalian circadian biology: elucidating genome-wide levels of temporal organization,” Annu Rev Genomics Hum Genet, Vol. 5, pp. 407-441 (2004)
Maemura, K. et al., “Molecular mechanisms of morning onset of myocardial infarction,” Ann N Y Acad Sci, Vol. 947, pp. 398-402 (2001)
Maemura, K. et al., “CLIF, a novel cycle-like factor, regulates the circadian oscillation of plasminogen activator inhibitor-1 gene expression,” J Biol. Chem., Vol. 275, no. 47, pp. 36847-51 (November 2000)
MacAulay, K. et al., “Use of lithium and SB-415286 to explore the role of glycogen synthase kinase-3 in the regulation of glucose transport and glycogen synthase,” Eur J Biochem, Vol. 270, pp. 3829-3838 (2003)
Mitchell, J. B. et al., “The immunophenotype of human adipose derived cells: Temporal changes in stromal- and stem cell-associated markers Stem Cells,”, Vol. 24, pp. 376-385 (2006)
Mitsui, S. et al., “Antagonistic role of E4BP4 and PAR proteins in the circadian oscillatory mechanism,” Genes Dev, Vol. 15, pp. 995-1006 (2001)
Mohri, T. et al., “Alterations of circadian expressions of clock genes in Dahl salt-sensitive rats fed a high-salt diet,” Hypertension, Vol. 42, no. 2, pp. 189-94 (August 2003)
Nagoshi, E. et al., “Circadian gene expression in individual fibroblasts: cell-autonomous and self-sustained oscillators pass time to daughter cells,” Cell, Vol. 119, pp. 693-705 (2004)
Naito, Y. et al., “Circadian gene expression of clock genes and plasminogen activator inhibitor-1 in heart and aorta of spontaneously hypertensive and Wistar-Kyoto rats,” J. Hypertens., Vol. 21, no. 6, pp. 1107-15 (June 2003)
Naito, Y. et al., “Augmented diurnal variations of the cardiac renin-angiotensin system in hypertensive rats,” Hypertension, Vol. 40, no. 6, pp. 827-33 (December 2002)
Narayanan, C. S. et al., “DBP binds to the proximal promoter and regulates liver-specific expression of the human angiotensinogen gene,” Biochem Biophys Res Commun., Vol. 25, no. 1, pp. 388-93 (October 1998)
Nelson, W. et al., “Methods for cosinor-rhythmometry,” Chronobiologia, Vol. 6, pp. 305-3²³ (1979)
Neuman, K. et al., “Helix-loop-helix transcription factors regulate Id2 gene promoter activity,” FEBS Lett, Vol. 374, pp. 279-283 (1995)
Oishi, K. et al., “Genome-wide expression analysis of mouse liver reveals CLOCK-regulated circadian output genes,” J Biol Chem, Vol. 278, pp. 41519-41527 (2003)
Oishi, K. et al., “Gene- and tissue-specific alterations of circadian clock gene expression in streptozotocin-induced diabetic mice under restricted feeding,” Biochem Biophys Res Commun, Vol. 317, pp. 330-334 (2004)
Panda, S. et al., “Circadian rhythms from flies to human,” Nature, Vol. 417, pp. 329-335 (2002a)
Panda, S. et al., “Coordinated transcription of key pathways in the mouse by the circadian clock,” Cell, Vol. 109, pp. 307-320 (2002b)
Patel, D. D. et al., “Disturbances in the normal regulation of SREBP-sensitive genes in PPAR alpha-deficient mice,” J Lipid Res., Vol. 42, no. 3, pp. 328-37 (March 2001)
Preitner, N. et al., “The orphan nuclear receptor REV-ERBalpha controls circadian transcription within the positive limb of the mammalian circadian oscillator,” Cell, Vol. 110, pp. 251-260 (2002)
Priestley, M. B., Spectral Analysis and Time Series, London: Academic Press (1981)
Rana, J. S. et al., “Circadian variation in the onset of myocardial infarction: effect of duration of diabetes,” Diabetes, Vol. 52, pp. 1464-1468 (2003)
Reick, M. et al., “NPAS2: an analog of clock operative in the mammalian forebrain,” Science, Vol. 293, pp. 506-509 (2001)
Richardson, V. M. et al., “Daily cycle of bHLH-PAS proteins, Ah receptor and Arnt, in multiple tissues of female Sprague-Dawley rats,” Biochem Biophys Res Commun, Vol. 252, pp. 225-231 (1998)
Ripperger, J. A. et al., “Circadian regulation of gene expression in animals,” Curr Opin Cell Biol, Vol. 13, pp. 357-362 (2001)
Ripperger, J. A. et al., “CLOCK, an essential pacemaker component, controls expression of the circadian transcription factor DBP,” Genes Dev, Vol. 14, pp. 679-689 (2000)
Rudic, R. D. et al., “BMAL1 and CLOCK, two essential components of the circadian clock, are involved in glucose homeostasis,” PLoS Biol, Vol. 2, pp. e377 (2004)
Schoenhard, J. A. et al., “Regulation of the PAI-1 promoter by circadian clock components: differential activation by BMAL1 and BMAL2,” J Mol Cell Cardiol., Vol. 35, no. 5, pp. 473-81 (May 2003)
Shimba, S. et al., “Brain and muscle Arnt-like protein-1 (BMAL1), a component of the molecular clock, regulates adipogenesis,” Proc Natl Acad Sci USA, Vol. 102, pp. 12071-12076 (2005)
Shimba, S. et al., “EPAS1 promotes adipose differentiation in 3T3-LI cells,” J Biol Chem, Vol. 279, pp. 40946-40953 (2004)
Stokkan, K. A. et al., “Entrainment of the circadian clock in the liver by feeding,” Science, Vol. 291, pp. 490-493 (2001)
Storch, K. F. et al., “Extensive and divergent circadian gene expression in liver and heart,” Nature, Vol. 417, pp. 78-83 (2002)
Stunkard, A. J. et al., “Two forms of disordered eating in obesity: binge eating and night eating,” Int J Obes Relat Metab Disord., Vol. 27, no. 1, pp. I-12 (January 2003)
Stunkard, A. J. et al., “The night-eating syndrome; a pattern of food intake among certain obese patients,” Am J Med, Vol. 19, pp. 78-86 (1955)
Theodoropoulou, M. et al., “Octreotide, a somatostatin analogue, mediates its antiproliferative action in pituitary tumor cells by altering phosphatidylinositol 3-kinase signaling and inducing Zac1 expression,” Cancer Res, Vol. 66, pp. 1576-82 (2006).
Turek, F. W. et al., “Obesity and metabolic syndrome in circadian Clock mutant mice,” Science, Vol. 308, pp. 1043-1045 (2005)
Ueda, H. R. et al., “A transcription factor response element for gene expression during circadian night,” Nature, Vol. 418, pp. 534-539 (2002)
van der Bom, J. G. et al., “The 4G5G polymorphism in the gene for PAI-1 and the circadian oscillation of plasma PAI-1,” Blood, Vol. 101, pp. 1841-1844 (2003)
Vgontzas, A. N. et al., “Adverse effects of modest sleep restriction on sleepiness, performance, and inflammatory cytokines,” J Clin Endocrinol Metab, Vol. 89, pp. 2119-2126 (2004)
Vgontzas, A. N. et al., “Chronic insomnia is associated with a shift of interleukin-6 and tumor necrosis factor secretion from nighttime to daytime,” Metabolism, Vol. 51, no. 7, pp. 887-892 (2002)
Vgontzas, A. N. et al., “Circadian interleukin-6 secretion and quantity and depth of sleep,” J Clin Endocrinol Metab., Vol. 84, no. 8, pp. 2603-2607 (1999)
Vgontzas, A. N. et al., “Marked decrease in sleepiness in patients with sleep apnea by etanercept, a tumor necrosis factor-alpha antagonist,” J Clin Endocrinol Metab, Vol. 89, pp. 4409-4413 (2004b)
Wichert, S. et al., “Identifying periodically expressed transcripts in microarray time series data,” Bioinformatics, Vol. 20, pp. 5-20 (2004)
Wilsbacher, L. D. et al., “Photic and circadian expression of luciferase in mPeriod1-luc transgenic mice in vivo,” Proc Natl Acad Sci USA, Vol. 99, pp. 489-494 (2002)
Yamaguchi, S. et al., “Role of DBP in the circadian oscillatory mechanism,” Mol Cell Biol, Vol. 20, pp. 4773-4781 (2000)
Yamazaki, S. et al., “Resetting central and peripheral circadian oscillators in transgenic rats,” Science, Vol. 288, pp. 682-685 (2000)
Yin, L. et al., “Nuclear receptor Rev-erbalpha is a critical lithium-sensitive component of the circadian clock,” Science, Vol. 311, pp. 1002-1005 (2006)
Yoo, S. H. et al., “PERIOD2:LUCIFERASE real-time reporting of circadian dynamics reveals persistent circadian oscillations in mouse peripheral tissues,” Proc Natl Acad Sci USA, Vol. 101, pp. 5339-5346 (2004)
Young, M. E. et al., “Alterations of the circadian clock in the heart by streptozotocin-induced diabetes,” J Mol Cell Cardiol, Vol. 34, pp. 223-231 (2002)
Young, M. E., “The circadian clock within the heart: potential influence on myocardial gene expression, metabolism, and function,” Am J Physiol Heart Circ Physiol, Vol. 290, pp. H1-16 (2006)

The complete disclosures of all references cited in this specification are hereby incorporated by reference. Also, incorporated by reference is the complete disclosure of the following documents, none of which are prior art to this application: S. Zvonic, et al., “Characterization of peripheral circadian clocks in adipose tissues,” Diabetes, vol. 55, pp. 962-970 (2006); A. A. Ptitsyn et al., “Circadian clocks are resounding in peripheral tissues,” PLoS Computational Biology, vol. 2, el 6, pp. 1-10 (2006); and X. Wu et al., “Circadian gene expression in human subcutaneous adipose-derived stem cells: Induction by dexamethasone, serum, and thiazolidinedione in the undifferentiated and adipogenic states,” a manuscript submitted to Endocrinology on May 31, 2006. In the event of an otherwise irreconcilable conflict, however, the present specification shall control.

What is claimed:

1. A method to increase weight gain in a mammal in need of weight gain, comprising administering to the mammal a therapeutically effective amount of a compound that lengthens the circadian gene expression in one or more peripheral clock genes in adipose tissue.

2. A method as in claim 1, wherein the peripheral clock genes are selected from a group consisting of BMAL1, Cry1, Cry2, Per1, Per2, Rev-erbα, Rev-erbβ, and Per3.

3. A method as in claim 1, wherein said compound is a compound known to inhibit glycogen synthase kinase 3 beta.

4. A method as in claim 1, wherein said compound is selected from the group consisting of lithium chloride, SB216763, SB415286, lithium chloride, insulin, phenylephrine, valproic acid, and histamine.

5. A method as in claim 1, wherein said mammal has diabetes, an immune dysfunctional disease, cachexia, anorexia nervosa, bipolar disorder, and Prater Willi Syndrome.

6. A method as in claim 1, wherein said compound is administered transdermally to subcutaneous adipose tissue.

7. A method as in claim 1, wherein said compound is injected directly into adipose tissue.

8. A method as in claim 1, where said compound is combined with a pharmacological acceptable carrier such that the combination is lipophilic.

9. A method to increase weight loss in a mammal in need of weight loss, comprising administering to the mammal a therapeutically effective amount of a compound that shortens the circadian gene expression in peripheral clock genes in adipose tissue.

10. A method as in claim 9, wherein the peripheral clock genes are selected from a group consisting of BMAL1, Cry1, Cry2, Per1, Per2, Rev-erbα, Rev-erbβ, and Per3.

11. A method as in claim 9, wherein said compound is a compound known to activate glycogen synthase kinase 3 beta.

12. A method as in claim 9, wherein said compound is selected from the group consisting of somatostatin, octreotide, somatostatin analogues, aspirin, and enzastaurin.

13. A method as in claim 9, wherein said mammal has diabetes, a weight disorder, metabolic syndrome.

14. A method as in claim 9, wherein said compound is administered transdermally to subcutaneous adipose tissue.

15. A method as in claim 9, wherein said compound is injected directly into adipose tissue.

16. A method as in claim 9, where said compound is combined with a pharmacological acceptable carrier such that the combination is lipophilic.

17. A method to screen compounds that are effective in modulating the circadian pattern of gene expression of certain peripheral clock genes in adipose cells, comprising obtaining adipose derived adult stem cells, exposing said cells to compounds to be tested for activity, obtaining RNA from said cells at multiple time periods, measuring the pattern of gene expression level as a function of time, and comparing the pattern of gene expression with the pattern of gene expression from adipose derived adult stem cells that were not exposed to the compound.

18. A method as in claim 9, wherein gene expression is measured from genes selected from a group consisting of BMAL1, Cry1, Cry2, Per1, Per2, Rev-erbα, Rev-erbβ, and Per3.
